(19) United States
(12) Patent Application Publication
(10) Pub. No.: US 2022/0273717 A1
(43) Pub. Date: Sep. 1, 2022

(54) IMPROVED FORMULATIONS FOR IMMUNE CELLS

(71) Applicant: Bellicum Pharmaceuticals, Inc., Houston, TX (US)

(72) Inventors: Omar Baez, Santa Isabel, PR (US); Devin Blass, Crestwood, KY (US)

(21) Appl. No.: 17/632,118

(22) PCT Filed: Aug. 12, 2020
(86) PCT No.: PCT/US20/45999

§ 371 Date: Feb. 1, 2022

Related U.S. Application Data
(60) Provisional application No. 62/885,747, filed on Aug. 12, 2019.

(51) Int. Cl. A61K 35/17; A61K 47/20; A61K 47/42; A61K 9/00; A61P 35/00

(52) U.S. Cl. CPC A61K 35/17; A61K 47/20; A61K 47/42; A61K 9/0019; A61P 35/00

(57) ABSTRACT

We disclose various improvements for compositions of T cells, including the use of lower levels of serum albumin and of cryoprotectants such as dimethyl sulfoxide.

### REFERENCE TO RELATED APPLICATIONS

This application claims the benefit of the priority date of U.S. provisional application 62/885,747, filed Aug. 12, 2019, the contents of which are incorporated herein in its entirety.

All documents and online information cited herein are incorporated by reference in their entirety.

### TECHNICAL FIELD

The invention is in the field of formulations for immune cells.

### BACKGROUND

T cell activation is an important step in the protective immunity against pathogenic microorganisms (e.g., viruses, bacteria, and parasites), foreign proteins, and harmful chemicals in the environment, and also as immunity against cancer and other hyperproliferative diseases.

T cells from a donor or a patient can be genetically-modified prior to their administration to the patient. For example, chimeric antigen receptor-expressing T cells may be used in various therapies, including different cancer therapies. Adoptive transfer of T cells expressing chimeric antigen receptors (CARs) is an effective therapy for the treatment of certain hematological malignancies. Modified T cells can also be used after haematopoietic cell transplantation (HCT) and stem cell transplantation (HSCT) to treat and prevent relapse and to provide immunity to the graft patients.

Genetically modified donor T cells are commonly obtained by taking T cells from the patient or a donor and shipping these to a facility where they are genetically engineered, frozen and returned to the treatment facility where the patient can then receive the therapy. Due to the requirement for the cells to be shipped and frozen, they are usually formulated together with additives such as cryoprotectants to protect the cells.

Known compositions have sometimes been associated with adverse effects in the recipients or high cost of manufacturing due to some of the additives present in the compositions. There is a need in the art to provide improved formulations which reduce the likelihood of adverse reactions and which can be manufactured in a cost effective manner.

### SUMMARY

The invention meets this need by providing compositions which comprises lower amounts of a cryoprotectant compared to prior art compositions, without any significant loss in cell viability or therapeutic efficacy even at high cell concentrations. These compositions are useful in therapy.

We provide a concentrated composition comprising T cells and serum albumin, wherein the composition comprises <2.5% (v/v) serum albumin (preferably <2.2% (v/v), <2% (v/v), or <1% (v/v), and preferably <0.5% (v/v) serum albumin) Such compositions achieve good therapeutic efficacy but are cheaper to prepare compared to prior art compositions which routinely comprise a higher amount of serum albumin, which is expensive in pharmaceutically-acceptable form.

The concentrated compositions preferably also include dimethyl sulfoxide (DMSO). DMSO will usually be present at an excess (v/v) compared to the serum albumin. Where the concentrated composition comprises x % (v/v) serum albumin, the concentration of DMSO (v/v) can thus be from 3x % to 30x %, and preferably the excess is from 3-fold to 15-fold e.g. from 6-fold to 12-fold, from 8-fold to 11-fold, or about 10-fold. An excess of at least 5-fold may be used e.g. from 5-fold to 3-fold.

Thus we also provide a concentrated composition comprising T cells, serum albumin, and DMSO, wherein the concentration of serum albumin (v/v) is from 0.1-2% (preferably <1% (v/v)) and the concentration of DMSO (v/v) is 3-fold to 30-fold higher than the concentration of serum albumin (preferably 3-fold to 15-fold higher e.g. from 6-fold to 12-fold higher, from 8-fold to 11-fold higher, or about 10-fold higher; a concentration which is at least 5-fold higher may be used).

The concentration of DMSO in these concentrated compositions is preferably <10% (v/v), and is more preferably from 2-8% (v/v), from 4-7% (v/v), from 5-6% (v/v), or about 5%.

We also provide a concentrated composition comprising T cells and DMSO, wherein the composition comprises <5% (v/v) DMSO. As discussed above, cryoprotectants have been reported to be associated with adverse reactions in the patient but were deemed important nonetheless to ensure inter alia good post-thaw viability of the cells. The inventors have shown that compositions comprising a lower amount of the cryoprotectant DMSO protect the cells well from thawing such that they show good post-thaw viability.

We also provide a concentrated composition comprising T cells, serum albumin, and DMSO, wherein the concentration of serum albumin (v/v) is 0.1-1% (e.g. 0.2-0.8%, 0.3-0.7%, 0.4-0.6%, or about 0.5%) and the concentration of DMSO is 5-7% (v/v). This range of DMSO concentrations has been found to provide good T cell recovery and viability, without requiring high levels of serum albumin.

These concentrated compositions may comprise between 1×10⁶ and 40×10⁶ T cells/mL, for example between 1×10⁶ and 35×10⁶, between 1.2×10⁶ and 33×10⁶, between 1.2×10⁶ and 30×10⁶, between 1.2×10⁶ and 20×10⁶, between 1.2×10⁶ and 15×10⁶, between 1.2×10⁶ and 10×10⁶, between 1.2×10⁶ and 5×10⁶ T cells/mL, or between 1.5×10⁶ and 2.5×10⁶ T cells/mL. A concentration of at least 1.5×10⁶ T cells/mL is typical, and a concentration of about 2×10⁶ T cells/mL is preferred. The concentration of T cells in YESCARTA™ is 3×10⁶/mL, and in KYMRIAH™ ranges from 2×10⁵/mL to 5×10⁶/mL.

All of the concentrated compositions can be provided in cryopreserved form (e.g. at a temperature of −80° C. or lower, and preferably −130° C. or lower) as well as in pre-cryopreserved and post-cryopreserved (e.g. thawed) forms.

In one aspect, the invention provides a container comprising a suspension of T cells, about 5-7% DMSO and about 0.1-1% (e.g. 0.2-0.8%, 0.3-0.7%, 0.4-0.6%, or about 0.5%) human albumin (v/v). In some embodiments, the container is a sterile infusion bag. In some embodiments, the infusion bag volume is about 15 mL, 30 mL, 50 mL, 60 mL, 70 mL, 100 mL, 250 mL, 500 mL, 750 mL, 1000 mL, 1500 mL, 2000 mL or 3000 mL.

Concentrated compositions of the invention can be administered to patients, but a high DMSO concentration can sometimes cause cellular damage, so the concentrated compositions can be diluted prior to administration to a patient. This dilution can help to avoid cellular damage. Diluted compositions are ideally administered to patients within 3 hours (e.g. within 60-120 minutes, and ideally within 90 minutes) of dilution to avoid even the diluted DMSO from causing cellular damage.

Thus we also provide a dilute composition comprising T cells and serum albumin, wherein the composition comprises <1% (v/v) serum albumin (preferably <0.6% (v/v), and more preferably 0.15% (v/v)). The dilute composition preferably also includes DMSO at an excess (v/v) compared to the serum albumin of from 3-fold to 30-fold, as described above (e.g. at least 5-fold excess DMSO).

Similarly, we provide a dilute composition comprising T cells, serum albumin, and DMSO, wherein the concentration of serum albumin (v/v) is from 0.03-0.6% (preferably <0.3% (v/v)) and the concentration of DMSO (v/v) is 3-fold to 30-fold higher than the concentration of serum albumin (preferably 3-fold to 15-fold higher e.g. from 6-fold to 12-fold higher, from 8-fold to 11-fold higher, or about 10-fold higher).

The concentration of DMSO in these dilute compositions is preferably <3% (v/v), and is more preferably from 0.6-2.4% (v/v), from 0.8-2.2% (v/v), from 1.5-2% (v/v), or from 1.6-1.9% (v/v), or about 1.77% (v/v).

We also provide a dilute composition comprising T cells, serum albumin, and DMSO, wherein the concentration of serum albumin is 0.03-0.3% (v/v) and the DMSO concentration is 0.15-0.20% (v/v).

These dilute compositions may comprise between 0.3×10⁶ and 12×10⁶ T cells/mL, for example between 0.3×10⁶ and 10×10⁶ T cells/mL, between 0.3×10⁶ and 12×10⁶ T cells/mL, between 0.3×10⁶ and 9×10⁶ T cells/mL, between 0.3×10⁶ and 7.5×10⁶ T cells/mL, between 0.3×10⁶ and 5×10⁶ T cells/mL, between 0.3×10⁶ and 2×10⁶ T cells/mL, between 0.3×10⁶ and 1.5×10⁶ T cells/mL, or between 0.5×10⁶ and 0.8×10⁶ T cells/mL. A concentration of at least 0.45×10⁶ T cells/mL is typical, and a concentration of about 0.6×10⁶ T cells/mL is preferred.

Dilute compositions can be (and are intended to be) administered to patients, so they are generally not frozen. Preferably, they will have a temperature of between 4° C. and 40° C., for example between 25-40° C., or at body temperature e.g. about 37+1° C. Compositions having a temperature between 5° C. and 15° C. may be typical after removal from a thawing water bath.

Further provided is a process for preparing a dilute composition as described herein, comprising the step of diluting a concentrated composition as described herein. Dilution may be performed with various liquids e.g. with an electrolyte solution (see below). This process may also involve a step of thawing a frozen concentrated composition, followed by the dilution. The diluted composition can then be administered to a patient (e.g. without a step of cell culture between dilution and administration).

The balance of a composition (i.e. in addition to the cellular, DMSO and serum albumin components) may be an electrolyte solution (see below). For example, cells may be prepared, then be washed and suspended (and optionally diluted e.g. after cryopreservation and thawing) in an electrolyte solution. Thus, a preferred composition may consist essentially of cells, DMSO, serum albumin, and an electrolyte solution.

The T cells in the concentrated and dilute compositions may be wild-type T cells (e.g. polyclonal T cells), but preferably they are genetically modified T cells (e.g. CAR-T cells). Genetically-modified T cells may express an activator switch and/or suicide switch. The T cells are most preferably human T cells.

The serum albumin in the compositions will generally be from the same species as the T cells in the composition. Most preferably, the T cells in the composition are human so, in a most preferred embodiment, the serum albumin is human serum albumin (HSA). This may be prepared from human serum or may be a recombinant human serum albumin.

Compositions of the invention are suitable and intended for administration to patients during therapy e.g. by intravenous infusion. Thus, a composition may be administered to a patient without further cell culture e.g. no cell culture medium is added to the composition prior to its administration. The recipient may have a hematological cancer (such as a treatment-refractory hematological cancer) or an inherited blood disorder. For instance, the recipient may have acute lymphoblastic leukemia (ALL), acute myeloid leukemia (AML), myelodysplastic syndrome (MDS), severe combined immune-deficiency (SCID), Wiskott-Aldrich syndrome (WA), Fanconi Anemia, chronic myelogenous leukemia (CML), non-Hodgkin lymphoma (NHL), Hodgkin lymphoma (HL), or multiple myeloma. The composition may help the recipient to control transplant-related infections following receipt of an allograft. The recipient may have received a hematopoietic stem cell transplant (HSCT). The T cells which are administered to a patient will usually be autologous T cells, and will usually have been genetically modified since being removed e.g. to express a CAR, a suicide switch, etc.

Thus, we also provide a method of treating a subject, comprising a step of administering a composition as described herein (preferably a dilute composition) to a patient in need thereof. Similarly, we provide the concentrated and dilute compositions, for use in treating a subject. In some embodiments, the method comprises a step of administering to a patient in need thereof the compositions described herein by intravenous infusion. In some embodiments, the intravenous infusion time is between 15 and 120 minutes. In some embodiments, the intravenous infusion time is up to 30 minutes. In some embodiments, the infusion volume is between 50 and 100 mL. In some embodiments, the infusion volume is about 50 mL.

### BRIEF DESCRIPTION OF DRAWINGS

FIG. 1 shows the prediction profiler for post-thaw cell viability.

FIG. 2 shows the prediction profiler for post-thaw cell recovery.

### DETAILED DESCRIPTION

### Formulations

Immune cells, such as T cells (in particular genetically modified T cells), are usually shipped to a patient's location in a frozen state. In order to retain viability during freezing and thawing, the cells are commonly formulated with cryoprotectants to protect them from damage. For example, the cryoprotectant dimethyl sulfoxide (DMSO) is added, usually at a concentration of 10% (v/v), to prevent the formation of ice crystals during the freezing process which could otherwise damage the cells.

Cryoprotectants such as DMSO have been associated with dose-dependent side effects, such as nausea, vomiting, abdominal cramps, headaches, hypotension, bradycardia, hemolysis, rashes, renal failure, hypertension, heart block, pulmonary edema, cardiac arrest, bronchospasm, and neurologic damage. There have therefore been attempts to reduce the concentration of DMSO to 5% (Morris et al. 2014 Transfusion 54:2514-22), but compositions still frequently contain 10% DMSO because reducing the concentration is difficult while preserving the cells.

In order to reduce the potential side effects on a recipient, treatment centers may therefore take measures to reduce the amount of DMSO before infusion, for example through washing the cells. However, this procedure is not ideal because cells can get lost in the process, and each additional handling step inherently increases the chances of contamination.

Serum albumin is also added to compositions to help in keeping T cells viable during the freeze/thaw cycle. The addition of serum albumin at certain concentrations is deemed essential to preserve the cells during freezing. For example, the formulation for KYMRIAH™ contains 20% (v/v) of 25% human serum albumin (HSA) and YESCARTA™ has 2.5% HSA. Reducing the amount of serum albumin would have the advantage of making the formulations more cost effective but known formulations nonetheless use high concentrations to ensure that the cells are not damaged whilst frozen.

There is no indication in the prior art that the concentrations of DMSO and serum albumin can both be reduced in these compositions without adversely affecting viability of the T cells.

The inventors have surprisingly discovered that T cells can be safely formulated using cryoprotectant concentrations which are lower than used in known formulations. These formulations are inter alia more cost effective and have additional safety features, but the T cells retain viability in the final compositions which is administered to the patient. This is achieved by providing the compositions described herein, with <2.5% (v/v) serum albumin Compositions in the art have been known to contain 2.5% (v/v) serum albumin (YESCARTA™) but others comprise much higher amounts of up to 5% (v/v), for example KYMRIAH™. The inventors have surprisingly discovered that the amount of serum albumin in the composition can be reduced without losing post-thaw viability of the T cells. In some embodiments, the composition comprises <2% (v/v), <1.5% (v/v), <1% (v/v) or even ≤0.5% (v/v) serum albumin (and in particular human serum albumin) The inventors have shown that such low concentrations achieve good post-thaw recovery of the cells. The level of serum albumin in the composition will usually be at least 0.05%.

Preferred compositions also include DMSO. DMSO may be present at a concentration of less than 10% (v/v). Lower concentrations may also be used and the inventors have seen particularly good results with concentrations in the range of 2 to 8% (v/v) of DMSO (example 2). Compositions of the invention may thus comprise between 2 and 8% (v/v), 4 and 7% (v/v), 4 and 6% (v/v) or between 5 and 6% (v/v) DMSO. Preferably, the composition comprises about 5.8% (v/v) of the cryoprotectant as the inventors have found this concentration to work particularly well.

Where a composition includes both serum albumin and DMSO, the DMSO can be present a concentration which is from 3-fold to 30-fold higher (v/v) than the serum albumin. The DMSO concentration can be from 3-fold to 15-fold higher than the serum albumin concentration, from 6-fold to 12-fold higher, from 8-fold to 11-fold higher, or about 10-fold higher. Known compositions such as KYMRIAH™ and YESCARTA™ have an excess of DMSO, but only 1.5× or 2×.

Compositions can optionally include cryoprotectants in addition to serum albumin and DMSO. For example the compositions can include ethylene glycol, glycerol, and/or diethylene glycol. Usually, however, these additional cryoprotectants are absent.

The compositions should be sterile. Ideally, they are also nonpyrogenic. They may be isotonic. The pH may be in the range of 6-8, for example in the range of 6.5-8, or of 6.8-7.7, for example about 7.4.

Electrolyte solutions may be used to prepare, wash or dilute compositions, and so compositions may include an electrolyte solution. The balance of a composition (i.e. in addition to the cellular, DMSO and serum albumin components) may be an electrolyte solution. Such electrolyte solutions are known in the art, such as the Plasma-Lyte™ products sold by Baxter Healthcare Corp. Plasma-Lyte™ products are a family of balanced crystalloid solutions which closely mimic human plasma in their content of electrolytes, osmolality and pH, and they are non-pyrogenic and isotonic. One suitable electrolyte solution has pH 6.5-8.0 and includes sodium, potassium, magnesium, acetate, chloride and gluconate e.g. including sodium chloride, sodium gluconate, sodium acetate, potassium chloride, and magnesium chloride. A preferred solution of this type is a sterile, nonpyrogenic, isotonic solution with a pH of 6.5-8.0 that has about 5.26 g/L NaCl, about 5.02 g/L sodium gluconate, about 3.68 g/L sodium acetate trihydrate, about 0.37 g/L KCl, and about 0.3 g/L MgCl₂.6H₂O (e.g. Plasma-Lyte A™). Another suitable electrolyte solution is a salt solution, such as a sodium chloride solution (e.g. normal saline, containing 9 g/L NaCl, usually with an osmolarity of 308 mOsm/L). These various solutions are ideally non-pyrogenic, isotonic, and sterile.

The concentrated compositions are preferably cryopreserved. This means that the composition will be frozen at a temperature of −80° C. or lower, and preferably −130° C. or lower.

The concentrated compositions are preferably in a container. In some embodiments, the container is a sterile infusion bag. In some embodiments, the infusion bag volume is about 15 mL, 30 mL, 50 mL, 60 mL, 70 mL, 100 mL, 250 mL, 500 mL, 750 mL, 1000 mL, 1500 mL, 2000 mL or 3000 mL.

Concentrated compositions may comprise between 1×10⁶ and 40×10⁶ cells/mL, for example between 1×10⁶ and 35×10⁶, between 1.2×10⁶ and 33×10⁶, between 1.2×10⁶ and 30×10⁶, between 1.2×10⁶ and 20×10⁶, between 1.2×10⁶ and 15×10⁶, between 1.2×10⁶ and 10×10⁶, between 1.2×10⁶ and 5×10⁶ T cells/mL, or between 1.5×10⁶ and 2.5×10⁶ T cells/mL. A concentration of about 2×10⁶ cells/mL may be preferred. These figures refer to the concentrations of desired T cells in the composition. The precise concentration can vary depending on the dose which is to be administered to the patient. Thus, where a higher cell number is to be administered to the patient, the cell concentration in the composition may be higher. Suitable cell concentrations can be determined by a clinician.

Before frozen concentrated compositions are administered to a patient they should be thawed. Methods for thawing therapeutic cells are well known in the art. For example, the frozen composition can be thawed using a water bath (e.g. at 37±1° C.). A sealed container (e.g. a cryopreservation bag, optionally sealed within a second container, such as a zip-seal plastic container) containing the cells can be submerged in a water bath to allow thawing to occur. The composition can be removed from the water bath before it reaches the same temperature as the water bath.

A thawed composition can be diluted before infusion into a patient, e.g., to prepare a diluted composition as described herein. These diluted compositions may comprise between 0.3×10⁶ and 12×10⁶ cells/mL, for example between 0.3×10⁶ and 12×10⁶ T cells/mL, between 0.3×10⁶ and 10×10⁶ T cells/mL, between 0.3×10⁶ and 9×10⁶ T cells/mL, between 0.3×10⁶ and 7.5×10⁶ T cells/mL, between 0.3×10⁶ and 5×10⁶ T cells/mL, between 0.3×10⁶ and 2×10⁶ T cells/mL, between 0.3×10⁶ and 1.5×10⁶ T cells/mL, or between 0.5×10⁶ and 0.8×10⁶ T cells/mL. A concentration of about 0.6×10⁶ cells/mL may be preferred. Again, these figures refer to the concentrations of desired T cells in the composition.

Compositions can include a pharmaceutically acceptable carrier. As used herein, the term “pharmaceutically acceptable” refers to a carrier that is compatible with the other ingredients of a pharmaceutical composition and can be safely administered to a subject. The term is used synonymously with “physiologically acceptable” and “pharmacologically acceptable”. Pharmaceutical compositions and techniques for their preparation and use are known to those of skill in the art in light of the present disclosure. For a detailed listing of suitable pharmacological compositions and techniques for their administration one may refer to texts such as Remington's Pharmaceutical Sciences, 17th ed. 1985; Brunton et al., “Goodman and Gilman's The Pharmacological Basis of Therapeutics,” McGraw-Hill, 2005; University of the Sciences in Philadelphia (eds.), “Remington: The Science and Practice of Pharmacy,” Lippincott Williams & Wilkins, 2005; and University of the Sciences in Philadelphia (eds.), “Remington: The Principles of Pharmacy Practice,” Lippincott Williams & Wilkins, 2008.

Pharmaceutically acceptable carriers will generally be sterile, at least for human use. A pharmaceutical composition will generally comprise agents for buffering and preservation in storage, and can include buffers and carriers for appropriate delivery, depending on the route of administration. Examples of pharmaceutically acceptable carriers include, without limitation, normal (0.9%) saline, phosphate-buffered saline (PBS) Hank's balanced salt solution (HBSS) and multiple electrolyte solutions such as PlasmaLyte A™ (Baxter). Pharmaceutically acceptable carriers can be adapted to the intended mode of administration. For example, compositions for intravenous injection can be sterile and include ingredients compatible with blood.

Formulations can be for immune cells, in particular, T cells, and other immune cells as well as other cells of the hematopoietic lineage such lymphoid or myeloid cells. In certain embodiments, the cells in a composition as disclosed herein consist of or consist essentially of a cell type or cell types named. For example, the composition can comprise cells, wherein the cells consist of, or consist essentially of, T cells. The term “consisting essentially of” refers to the inclusion of recited elements and other elements that do not materially affect the basic and novel characteristics of a claimed combination.

Methods for diluting T cell compositions are known in the art. For example, the composition may be diluted using an electrolyte solution as discussed herein e.g. using a Plasma-Lyte™ solution such as a Plasma-Lyte A™ solution, or using a saline solution. After dilution a composition is ideally administered to a subject within 90 minutes.

Dilution can be at any ratio deemed suitable by a clinician. For example, the composition may be diluted at a ratio of between 1:2 and 1:10. Preferably, the composition is diluted at a ratio of 3:7 (i.e. 1:2⅓). The KYMRIAH™ and YESCARTA™ products are not diluted prior to infusion.

In some embodiments, the composition will be diluted such that it contains a desired cell concentration after dilution. To this end, the cell number in the composition can be determined and a dilution factor calculated such that the final diluted composition contains a desired number of cells/mL.

Concentrated compositions can have various volumes. Typical volumes will be between 5-100 mL, for example between 10-20 mL (e.g. about 15 mL) or between 50-70 mL (e.g. about 60 mL). In some embodiments the composition has a volume of at least 14 mL; in some embodiments a cryopreserved composition has a volume of at least 2 mL e.g. at least 5 mL. A volume of about 15 mL may be useful for compositions including fewer than 500×10⁶ cells, whereas a volume of about 60 mL may be useful for compositions comprising more than 500×10⁶ cells.

Compositions can be located within a cryopreservation bag, which may in turn be located within a cryopreservation cassette or within a zip-seal plastic container. Concentrated compositions within a cryopreservation bag may be diluted within the bag to give a diluted composition e.g. by introducing an electrolyte solution via syringe. For instance, to give a 7:3 dilution ratio, 35 mL of electrolyte solution may be added to 15 mL of a concentrated composition, or 140 mL of electrolyte solution may be added to 60 mL of a concentrated composition.

A cryopreservation bag (or other container) preferably includes an identification label which conforms to the ISBT 128 standard (as approved in 1994). Typical cryopreservation bags include an exit port, which can be a spike port having a septum, and which can be protected from external contamination by a tamper-evident cover.

A cryopreservation can include a spike port configured to accept a bag spike for withdrawing material from the bag, e.g, through a tube.

In some embodiments, compositions of the invention are substantially free from phenol red (phenolsulfonphthalein) e.g. the concentration of phenol red is less than 50 μg/L, and ideally less than 5 μg/L. Residual phenol red may be present, for instance, after use of RPMI-1640 medium. Compositions may contain no phenol red.

We provide a concentrated composition comprising human T cells and human serum albumin, wherein the composition comprises (i) from 0.5% (v/v) to 2% (v/v) human serum albumin, and (ii) from 1.5×10⁶ T cells/mL to 40×10⁶ T cells/mL. This composition can also include from 2-10% (v/v) DMSO.

We provide a concentrated composition comprising human T cells and human serum albumin, wherein the composition comprises (i) from 0.5% (v/v) to 2% (v/v) human serum albumin, and (ii) from 1.5×10⁶ T cells/mL to 35×10⁶ T cells/mL. This composition can also include from 2-10% (v/v) DMSO.

We provide a concentrated composition comprising human T cells and human serum albumin, wherein the composition comprises (i) from 0.5% (v/v) to 2% (v/v) human serum albumin, and (ii) from 1.5×10⁶ T cells/mL to 30×10⁶ T cells/mL. This composition can also include from 2-10% (v/v) DMSO.

We provide a concentrated composition comprising human T cells and human serum albumin, wherein the composition comprises (i) from 0.5% (v/v) to 2% (v/v) human serum albumin, and (ii) from 1.5×10⁶ T cells/mL to 20×10⁶ T cells/mL. This composition can also include from 2-10% (v/v) DMSO.

We provide a concentrated composition comprising human T cells and human serum albumin, wherein the composition comprises (i) from 0.5% (v/v) to 2% (v/v) human serum albumin, and (ii) from 1.5×10⁶ T cells/mL to 10×10⁶ T cells/mL. This composition can also include from 2-10% (v/v) DMSO.

We provide a concentrated composition comprising human T cells and DMSO, wherein the composition comprises (i) from 2-8% (v/v) DMSO, and (ii) from 1.5×10⁶ T cells/mL to 40×10⁶ T cells/mL. This composition can also include <2% (v/v) human serum albumin, and preferably from 0.4-1% (v/v) human serum albumin.

We provide a concentrated composition comprising human T cells and DMSO, wherein the composition comprises (i) from 2-8% (v/v) DMSO, and (ii) from 1.5×10⁶ T cells/mL to 35×10⁶ T cells/mL. This composition can also include <2% (v/v) human serum albumin, and preferably from 0.4-1% (v/v) human serum albumin.

We provide a concentrated composition comprising human T cells and DMSO, wherein the composition comprises (i) from 2-8% (v/v) DMSO, and (ii) from 1.5×10⁶ T cells/mL to 30×10⁶ T cells/mL. This composition can also include <2% (v/v) human serum albumin, and preferably from 0.4-1% (v/v) human serum albumin.

We provide a concentrated composition comprising human T cells and DMSO, wherein the composition comprises (i) from 2-8% (v/v) DMSO, and (ii) from 1.5×10⁶ T cells/mL to 20×10⁶ T cells/mL. This composition can also include <2% (v/v) human serum albumin, and preferably from 0.4-1% (v/v) human serum albumin.

We provide a concentrated composition comprising human T cells and DMSO, wherein the composition comprises (i) from 2-8% (v/v) DMSO, and (ii) from 1.5×10⁶ T cells/mL to 10×10⁶ T cells/mL. This composition can also include <2% (v/v) human serum albumin, and preferably from 0.4-1% (v/v) human serum albumin.

We provide a concentrated composition comprising human T cells, DMSO, and human serum albumin, wherein (i) the composition comprises from 1.5×10⁶ T cells/mL to 40×10⁶ T cells/mL, (ii) the composition comprises 0.5% (v/v) to 2% (v/v) human serum albumin, and (iii) the concentration of DMSO is from 5-15-fold higher, preferably about 10-fold higher, than the concentration of human serum albumin.

We provide a concentrated composition comprising human T cells, DMSO, and human serum albumin, wherein (i) the composition comprises from 1.5×10⁶ T cells/mL to 35×10⁶ T cells/mL, (ii) the composition comprises 0.5% (v/v) to 2% (v/v) human serum albumin, and (iii) the concentration of DMSO is from 5-15-fold higher, preferably about 10-fold higher, than the concentration of human serum albumin.

We provide a concentrated composition comprising human T cells, DMSO, and human serum albumin, wherein (i) the composition comprises from 1.5×10⁶ T cells/mL to 30×10⁶ T cells/mL, (ii) the composition comprises 0.5% (v/v) to 2% (v/v) human serum albumin, and (iii) the concentration of DMSO is from 5-15-fold higher, preferably about 10-fold higher, than the concentration of human serum albumin.

We provide a concentrated composition comprising human T cells, DMSO, and human serum albumin, wherein (i) the composition comprises from 1.5×10⁶ T cells/mL to 20×10⁶ T cells/mL, (ii) the composition comprises 0.5% (v/v) to 2% (v/v) human serum albumin, and (iii) the concentration of DMSO is from 5-15-fold higher, preferably about 10-fold higher, than the concentration of human serum albumin.

We provide a concentrated composition comprising human T cells, DMSO, and human serum albumin, wherein (i) the composition comprises from 1.5×10⁶ T cells/mL to 10×10⁶ T cells/mL, (ii) the composition comprises 0.5% (v/v) to 2% (v/v) human serum albumin, and (iii) the concentration of DMSO is from 5-15-fold higher, preferably about 10-fold higher, than the concentration of human serum albumin.

We provide a concentrated composition comprising human T cells and human serum albumin, wherein the composition comprises (i) <1% (v/v), and preferably about 0.5% (v/v) human serum albumin, and (ii) from 1.5×10⁶ T cells/mL to 5×10⁶ T cells/mL, and preferably about 2×10⁶ T cells/mL. This composition can also include <10% (v/v) DMSO, and preferably about 5% (v/v) DMSO.

We provide a concentrated composition comprising human T cells, DMSO, and human serum albumin, wherein the composition comprises (i) <1% (v/v), and preferably about 0.5% (v/v) human serum albumin, (ii) from 1.5×10⁶ T cells/mL to 5×10⁶ T cells/mL, and preferably about 2×10⁶ T cells/mL, and (iii) <10% (v/v) DMSO, and preferably about 5% (v/v) DMSO.

We provide a concentrated composition comprising human T cells and DMSO, wherein the composition comprises (i) <8% (v/v) DMSO, and preferably about 5% (v/v) DMSO, and (ii) from 1.5×10⁶ T cells/mL to 5×10⁶ T cells/mL, and preferably about 2×10⁶ T cells/mL. This composition can also include <1% (v/v) human serum albumin, and preferably about 0.5% (v/v) human serum albumin.

We provide a concentrated composition comprising from 1.5×10⁶ to 40×10⁶ genetically modified human T cells per milliliter, about 0.5% (v/v) human serum albumin, and about 5% (v/v) DMSO. For instance, we provide a composition comprising about 30×10⁶ genetically modified human T cells per milliliter, about 0.5% (v/v) human serum albumin, and about 5% (v/v) DMSO.

We provide a concentrated composition comprising from 1.5×10⁶ to 30×10⁶ genetically modified human T cells per milliliter, about 0.5% (v/v) human serum albumin, and about 5% (v/v) DMSO. For instance, we provide a composition comprising about 20×10⁶ genetically modified human T cells per milliliter, about 0.5% (v/v) human serum albumin, and about 5% (v/v) DMSO.

We provide a concentrated composition comprising from 1.5×10⁶ to 20×10⁶ genetically modified human T cells per milliliter, about 0.5% (v/v) human serum albumin, and about 5% (v/v) DMSO. For instance, we provide a composition comprising about 12×10⁶ genetically modified human T cells per milliliter, about 0.5% (v/v) human serum albumin, and about 5% (v/v) DMSO.

We provide a concentrated composition comprising from 1.5×10⁶ to 10×10⁶ genetically modified human T cells per milliliter, about 0.5% (v/v) human serum albumin, and about 5% (v/v) DMSO. For instance, we provide a composition comprising about 2×10⁶ genetically modified human T cells per milliliter, about 0.5% (v/v) human serum albumin, and about 5% (v/v) DMSO.

We provide a concentrated composition comprising human T cells, DMSO, and human serum albumin, wherein (i) the composition comprises from 1.5×10⁶ T cells/mL to 5×10⁶ T cells/mL, and preferably about 2×10⁶ T cells/mL (ii) the composition comprises <1% (v/v), and preferably about 0.5% (v/v) human serum albumin, and (iii) the concentration of DMSO is from 5-15-fold higher, preferably about 10-fold higher, than the concentration of human serum albumin.

We provide a concentrated composition comprising human T cells, human serum albumin, and DMSO, wherein the concentration of human serum albumin (v/v) is from 0.4-0.6%, or about 0.5%, the concentration of DMSO is 5-7% (v/v), and the concentration of human T cells is from 1.5×10⁶ to 40×10⁶ T cells/mL, preferably about 30×10⁶ T cells/mL, preferably about 20×10⁶ T cells/mL, preferably about 15×10⁶ T cells/mL, preferably about 10×10⁶ T cells/mL, preferably about 5×10⁶ T cells/mL, preferably about 2×10⁶ T cells/mL.

We provide a dilute composition comprising human T cells and human serum albumin, wherein the composition comprises (i) from 0.15% (v/v) to 0.6% (v/v) human serum albumin, and (ii) from 0.5×10⁶ T cells/mL to 12×10⁶ T cells/mL, for example between 0.3×10⁶ and 10×10⁶ T cells/mL, between 0.3×10⁶ and 9×10⁶ T cells/mL, between 0.3×10⁶ and 7.5×10⁶ T cells/mL, between 0.3×10⁶ and 5×10⁶ T cells/mL, between 0.3×10⁶ and 2×10⁶ T cells/mL, between 0.3×10⁶ and 1.5×10⁶ T cells/mL, or between 0.5×10⁶ and 0.8×10⁶ T cells/mL. This composition can also include from 0.6-3% (v/v) DMSO.

We provide a dilute composition comprising human T cells and human serum albumin, wherein the composition comprises (i) from 0.15% (v/v) to 0.6% (v/v) human serum albumin, and (ii) from 0.5×10⁶ T cells/mL to 3×10⁶ T cells/mL. This composition can also include from 0.6-3% (v/v) DMSO.

We provide a dilute composition comprising human T cells and DMSO, wherein the composition comprises (i) from 0.6-2.5% (v/v) DMSO, and (ii) from 0.5×10⁶ T cells/mL to 12×10⁶ T cells/mL, for example between 0.3×10⁶ and 10×10⁶ T cells/mL, between 0.3×10⁶ and 9×10⁶ T cells/mL, between 0.3×10⁶ and 7.5×10⁶ T cells/mL, between 0.3×10⁶ and 5×10⁶ T cells/mL, between 0.3×10⁶ and 2×10⁶ T cells/mL, between 0.3×10⁶ and 1.5×10⁶ T cells/mL, or between 0.5×10⁶ and 0.8×10⁶ T cells/mL. This composition can also include from 0.6-3% (v/v) DMSO. This composition can also include <0.6% (v/v) human serum albumin, and preferably from 0.1-0.3% (v/v) human serum albumin.

We provide a dilute composition comprising human T cells and DMSO, wherein the composition comprises (i) from 0.6-2.5% (v/v) DMSO, and (ii) from 0.5×10⁶ T cells/mL to 3×10⁶ T cells/mL. This composition can also include from 0.6-3% (v/v) DMSO. This composition can also include <0.6% (v/v) human serum albumin, and preferably from 0.1-0.3% (v/v) human serum albumin.

We provide a dilute composition comprising human T cells, DMSO, and human serum albumin, wherein (i) the composition comprises from 0.5×10⁶ T cells/mL to 12×10⁶ T cells/mL, for example between 0.3×10⁶ and 10×10⁶ T cells/mL, between 0.3×10⁶ and 9×10⁶ T cells/mL, between 0.3×10⁶ and 7.5×10⁶ T cells/mL, between 0.3×10⁶ and 5×10⁶ T cells/mL, between 0.3×10⁶ and 2×10⁶ T cells/mL, between 0.3×10⁶ and 1.5×10⁶ T cells/mL, or between 0.5×10⁶ and 0.8×10⁶ T cells/mL, (ii) the composition comprises 0.15% (v/v) to 0.6% (v/v) human serum albumin, and (iii) the concentration of DMSO is from 5-15-fold higher, preferably about 10-fold higher, than the concentration of human serum albumin.

We provide a dilute composition comprising human T cells, DMSO, and human serum albumin, wherein (i) the composition comprises from 0.5×10⁶ T cells/mL to 3×10⁶ T cells/mL, (ii) the composition comprises 0.15% (v/v) to 0.6% (v/v) human serum albumin, and (iii) the concentration of DMSO is from 5-15-fold higher, preferably about 10-fold higher, than the concentration of human serum albumin.

We provide a dilute composition comprising human T cells and human serum albumin, wherein the composition comprises (i) <0.3% (v/v), and preferably about 0.15% (v/v) human serum albumin, and (ii) from 0.5×10⁶ T cells/mL to 1.5×10⁶ T cells/mL, and preferably about 0.6×10⁶ T cells/mL. This composition can also include <3% (v/v) DMSO, and preferably about 1.5% (v/v) DMSO.

We provide a dilute composition comprising human T cells, DMSO, and human serum albumin, wherein the composition comprises (i) <0.3% (v/v), and preferably about 0.15% (v/v) human serum albumin, (ii) from 0.5×10⁶ T cells/mL to 1.5×10⁶ T cells/mL, and preferably about 0.6×10⁶ T cells/mL, and (iii) <3% (v/v) DMSO, and preferably about 1.5% (v/v) DMSO.

We provide a dilute composition comprising human T cells and DMSO, wherein the composition comprises (i) <2.5% (v/v) DMSO, and preferably about 1.5% (v/v) DMSO, and (ii) from 0.5×10⁶ T cells/mL to 1.5×10⁶ T cells/mL, and preferably about 0.6×10⁶ T cells/mL. This composition can also include <0.3% (v/v) human serum albumin, and preferably about 0.15% (v/v) human serum albumin.

We provide a concentrated composition comprising from 0.5×10⁶ to 3×10⁶ genetically modified human T cells per milliliter, about 0.5% (v/v) human serum albumin, and about 5% (v/v) DMSO. For instance, we provide a composition comprising about 0.6×10⁶ genetically modified human T cells per milliliter, about 0.15% (v/v) human serum albumin, and about 1.5% (v/v) DMSO.

We provide a dilute composition comprising human T cells, DMSO, and human serum albumin, wherein (i) the composition comprises from 0.5×10⁶ T cells/mL to 1.5×10⁶ T cells/mL, and preferably about 0.6×10⁶ T cells/mL (ii) the composition comprises <0.3% (v/v), and preferably about 0.15% (v/v) human serum albumin, and (iii) the concentration of DMSO is from 5-15-fold higher, preferably about 10-fold higher, than the concentration of human serum albumin.

We provide a dilute composition comprising human T cells, human serum albumin, and DMSO, wherein the concentration of human serum albumin (v/v) is from 0.4-0.6%, or about 0.5%, the concentration of DMSO is 5-7% (v/v), and the concentration of human T cells is from 1.5×10⁶ to 5×10⁶ cells/mL, preferably about 2×10⁶ cells/mL.

In some embodiments, the compositions described herein comprising T cells are supplied in an infusion bag containing approximately 15 to 60 mL of frozen suspension of T cells in 5-7% DMSO and 0.1-1% human serum albumin. In some embodiments, the T cells are supplied in an infusion bag containing approximately 15-100 mL, 15-90 mL, 15-80 mL, 15-70 mL, 15-60 mL, 60-70 mL, 60-75 mL, or 65-75 mL, of suspension of T cells in 5-7% DMSO and 0.1-1% human serum albumin.

Concentrations of serum albumin are expressed herein in terms of v/v because in practice the serum albumin is incorporated by volumetric dilution of a concentrated serum albumin solution (for instance, 1% HSA can be achieved by a 25 fold volumetric dilution of a 25% solution e.g. diluting 40 mL into a final volume of 1 L), but the concentrated serum albumin solution will have been prepared on a weight basis (e.g. a concentrated 25% solution contains 25 g of serum albumin in an aqueous volume of 100 mL). Ultimately, therefore, a v/v percentage of serum albumin reflects in practice a w/v percentage on a 1:1 basis i.e. these units are used interchangeably.

### T Cells

The compositions disclosed herein comprise T cells. Examples of suitable T cells for use with the invention include CAR-T cells and wild-type polyclonal T cells. The compositions are particularly useful for genetically modified T cells, such as CAR-T cells.

T cells which are genetically modified are useful for administration to subjects who can benefit from donor lymphocyte administration. These subjects will typically be humans. Most preferably, T cells are therefore human T cells.

The T cells in a composition of the invention will generally be derived from a single donor, and they may be intended for reintroduction to that donor or to another recipient, e.g., after expansion and/or genetic modification.

The T cells can be derived from any donor. The donor will generally be an adult (at least 18 years old) but children are also suitable as T cell donors (e.g. see Styczynski 2018, Transfus Apher Sci 57(3):323-330). The T cells can also be derived from the same patients in need of the genetically modified cells.

A suitable process for obtaining T cells from a donor is described in the published protocol which accompanied Di Stasi et al. (2011) N Engl J Med 365:1673-83. In general terms, T cells are obtained from a donor, subjected to genetic modification and selection, and can then be administered to recipient subjects. A useful source of T cells is the donor's peripheral blood. Peripheral blood samples will generally be subjected to leukapheresis to provide a sample enriched for white blood cells. This enriched sample (also known as a leukopak) can be composed of a variety of blood cells including monocytes, lymphocytes, platelets, plasma, and red cells. A leukopak typically contains a higher concentration of cells as compared to venipuncture or buffy coat products.

Although the sample may be subjected to allodepletion, it is preferred that the sample is not subjected to allodepletion. Preferred samples are thus alloreplete, as discussed in Zhou et al. (2015) Blood 125:4103-13. These populations provide a more robust T cell repertoire for providing the therapeutic advantages of the donor cells. Preferred compositions of the invention are thus not T cell allodepleted, and have not been subject to a step of allodepletion.

Donor T cells are generally cultured (usually under activating conditions e.g. using anti-CD3 and/or anti-CD28 antibodies, optionally in the presence of cytokine such as IL-2, IL-15, and/or IL-7) prior to being genetically modified. This step provides higher yields of T cells at the end of the modification process.

The T cells can be transduced using a viral vector encoding a desirable nucleic acid such as a safety switch of interest, a costimulatory switch and/or a chimeric antigen receptors (see below). Suitable transduction techniques are disclosed but also well known in the art and may involve fibronectin fragment CH-296. As an alternative to transduction using a viral vector, cells can be transfected with DNA encoding the nucleic acid of interest and a cell surface transgene marker of interest e.g. using calcium phosphate, cationic polymers (such as PEI), magnetic beads, electroporation and commercial lipid-based reagents such as Lipofectamine™ and Fugene™. One result of the transduction/transfection step is that various donor T cells will now be genetically-modified T cells which can express the suicide switch of interest.

A preferred viral vector for transduction is the retroviral vector disclosed by Tey et al. (2007) Biol Blood Marrow Transpl 13:913-24 and by Di Stasi et al. (2011) supra. This vector is based on Gibbon ape leukemia virus (Gal-V) pseudotyped retrovirus encoding an iCasp9 suicide switch and a ΔCD19 cell surface transgene marker (see further below). It can be produced in the PG13 packaging cell line, as discussed by Tey et al. (2007) supra. Other viral vectors encoding the desired proteins can also be used. Retroviral vectors are preferred, particularly those which can provide a high copy number of proviral integrants per cell.

After transduction/transfection, cells can be separated from transduction/transfection materials and cultured again, to permit the genetically-modified T cells to expand. T cells can be expanded so that a desired minimum number of genetically-modified T cells is achieved.

A particular adoptive cell transfer method uses CAR-modified T cells and holds great promise for the treatment of a variety of malignancies. In this therapy, T cells are extracted from a patient's or donor's blood and genetically engineered to express chimeric antigen receptors (CARs) on the cell surface.

### Chimeric Antigen Receptors

In some embodiments, the compositions and methods described herein use a CAR-T cell population. By “chimeric antigen receptor” or “CAR” is meant, for example, a chimeric polypeptide that comprises a polypeptide sequence that recognizes a target antigen (an antigen-recognition domain, antigen recognition region, antigen recognition moiety, or antigen binding region) linked to a transmembrane polypeptide and intracellular domain polypeptide selected to activate the T cell and provide specific immunity.

### Antigen Recognition Moieties

An “antigen recognition moiety” may be any polypeptide or fragment thereof, such as, for example, an antibody fragment variable domain, either naturally derived, or synthetic, which binds to an antigen. Examples of antigen recognition moieties include, but are not limited to, polypeptides derived from antibodies, such as, for example, single chain variable fragments (scFv), Fab, Fab′, F(ab′)2, and Fv fragments; polypeptides derived from T Cell receptors, such as, for example, TCR variable domains; secreted factors (e.g., cytokines, growth factors) that can be artificially fused to signaling domains (e.g., “zytokines”), and any ligand or receptor fragment (e.g., CD27, NKG2D) that binds to the extracellular cognate protein. Combinatorial libraries could also be used to identify peptides binding with high affinity to tumor-associated targets. Moreover, “universal” CARs can be made by fusing aviden to the signaling domains in combination with biotinylated tumor-targeting antibodies (Urbanska (12) Ca Res) or by using Fc gamma receptor/CD16 to bind to IgG-targeted tumors (Kudo K (13) Ca Res).

### Transmembrane Regions

A chimeric protein herein may include a single-pass or multiple pass transmembrane sequence (e.g., at the N-terminus or C-terminus of the chimeric protein). Single pass transmembrane regions are found in certain CD molecules, tyrosine kinase receptors, serine/threonine kinase receptors, TGFβ, BMP, activin and phosphatases. Single pass transmembrane regions often include a signal peptide region and a transmembrane region of about 20 to about 25 amino acids, many of which are hydrophobic amino acids and can form an alpha helix. A short track of positively charged amino acids often follows the transmembrane span to anchor the protein in the membrane. Multiple pass proteins include ion pumps, ion channels, and transporters, and include two or more helices that span the membrane multiple times. All or substantially all of a multiple pass protein sometimes is incorporated in a chimeric protein. Sequences for single pass and multiple pass transmembrane regions are known and can be selected for incorporation into a chimeric protein molecule.

In some embodiments, the transmembrane domain is fused to the extracellular domain of the CAR. In one embodiment, the transmembrane domain that naturally is associated with one of the domains in the CAR is used. In other embodiments, a transmembrane domain that is not naturally associated with one of the domains in the CAR is used. In some instances, the transmembrane domain can be selected or modified by amino acid substitution (e.g., typically charged to a hydrophobic residue) to avoid binding of such domains to the transmembrane domains of the same or different surface membrane proteins to minimize interactions with other members of the receptor complex.

Transmembrane domains may, for example, be derived from the alpha, beta, or zeta chain of the T cell receptor, CD3-c, CD3 CD4, CD5, CD8, CD8a, CD9, CD16, CD22, CD28, CD33, CD38, CD64, CD80, CD86, CD134, CD137, or CD154. Or, in some examples, the transmembrane domain may be synthesized de novo, comprising mostly hydrophobic residues, such as, for example, leucine and valine. In certain embodiments a short polypeptide linker may form the linkage between the transmembrane domain and the intracellular domain of the chimeric antigen receptor. The chimeric antigen receptors may further comprise a stalk, that is, an extracellular region of amino acids between the extracellular domain and the transmembrane domain. For example, the stalk may be a sequence of amino acids naturally associated with the selected transmembrane domain. In some embodiments, the chimeric antigen receptor comprises a CD8 transmembrane domain, in certain embodiments, the chimeric antigen receptor comprises a CD8 transmembrane domain, and additional amino acids on the extracellular portion of the transmembrane domain, in certain embodiments, the chimeric antigen receptor comprises a CD8 transmembrane domain and a CD8 stalk. The chimeric antigen receptor may further comprise a region of amino acids between the transmembrane domain and the cytoplasmic domain, which are naturally associated with the polypeptide from which the transmembrane domain is derived.

### Target Antigens

Chimeric antigen receptors bind to target antigens. When assaying T cell activation in vitro or ex vivo, target antigens may be obtained or isolated from various sources. The target antigen, as used herein, is an antigen or immunological epitope on the antigen, which is crucial in immune recognition and ultimate elimination or control of the disease-causing agent or disease state in a mammal. The immune recognition may be cellular and/or humoral. In the case of intracellular pathogens and cancer, immune recognition may, for example, be a T lymphocyte response.

The target antigen may be derived or isolated from, for example, a pathogenic microorganism such as viruses including HIV (Korber et al, eds HIV Molecular Immunology Database, Los Alamos National Laboratory, Los Alamos, N. Mex), influenza, Herpes simplex, human papilloma virus (U.S. Pat. No. 5,719,054), Hepatitis B (U.S. Pat. No. 5,780,036), Hepatitis C (U.S. Pat. No. 5,709,995), EBV, Cytomegalovirus (CMV) and the like. Target antigen may be derived or isolated from pathogenic bacteria such as, for example, from Chlamydia (U.S. Pat. No. 5,869,608), Mycobacteria, Legionella, Meningococcus, Group A Streptococcus, Salmonella, Listeria, Hemophilus influenzae (U.S. Pat. No. 5,955,596) and the like). Target antigen may be derived or isolated from, for example, pathogenic yeast including Aspergillus, invasive Candida (U.S. Pat. No. 5,645,992), Nocardia, Histoplasmosis, Cryptosporidia and the like. Target antigen may be derived or isolated from, for example, a pathogenic protozoan and pathogenic parasites including but not limited to Pneumocystis carinii, Trypanosoma, Leishmania (U.S. Pat. No. 5,965,242), Plasmodium (U.S. Pat. No. 5,589,343) and Toxoplasma gondii.

The term “antigen” as used herein is defined as a molecule that provokes an immune response. This immune response may involve either antibody production, or the activation of specific immunologically competent cells, or both. An antigen can be derived from organisms, subunits of proteins/antigens, killed or inactivated whole cells or lysates. Therefore, any macromolecules, including virtually all proteins or peptides, can serve as antigens. Furthermore, antigens can be derived from recombinant or genomic DNA, including, for example, any DNA that contains nucleotide sequences or partial nucleotide sequences of a pathogenic genome or a gene or a fragment of a gene for a protein that elicits an immune response results in synthesis of an antigen.

Target antigen includes an antigen associated with a preneoplastic or hyperplastic state. Target antigen may also be associated with, or causative of cancer. Such target antigen may be, for example, tumor specific antigen, tumor associated antigen (TAA) or tissue specific antigen, epitope thereof, and epitope agonist thereof. Such target antigens include but are not limited to carcinoembryonic antigen (CEA) and epitopes thereof such as CAP-1, CAP-1-6D and the like (GenBank Accession No. M29540), MART-1 (Kawakarni et al, J. Exp. Med. 180:347-352, 1994), MAGE-1 (U.S. Pat. No. 5,750,395), MAGE-3, GAGE (U.S. Pat. No. 5,648,226), GP-100 (Kawakami et al Proc. Nat'l Acad. Sci. USA 91:6458-6462, 1992), MUC-1, MUC-2, point mutated ras oncogene, normal and point mutated p53 oncogenes (Hollstein et al Nucleic Acids Res. 22:3551-3555, 1994), PSMA (Israeli et al Cancer Res. 53:227-230, 1993), tyrosinase (Kwon et al PNAS 84:7473-7477, 1987) TRP-1 (gp75) (Cohen et al Nucleic Acid Res. 18:2807-2808, 1990; U.S. Pat. No. 5,840,839), NY-ESO-1 (Chen et al PNAS 94: 1914-1918, 1997), TRP-2 (Jackson et al EMBOJ, 11:527-535, 1992), TAG72, KSA, CA-125, CD-123, PSA, HER-2/neu/c-erb/B2, (U.S. Pat. No. 5,550,214), BRC-I, BRC-II, bcr-abl, pax3-fkhr, ews-fli-1, modifications of TAAs and tissue specific antigen, splice variants of TAAs, epitope agonists, and the like. Other TAAs may be identified, isolated and cloned by methods known in the art such as those disclosed in U.S. Pat. No. 4,514,506. Target antigen may also include one or more growth factors and splice variants of each. A tumor antigen is any antigen such as, for example, a peptide or polypeptide, that triggers an immune response in a host against a tumor. The tumor antigen may be a tumor-associated antigen, which is associated with a neoplastic tumor cell.

scFvs that target these antigens are known in the art, see, e.g., Berahovich et al., 2018 Cancers 10, 323; Wang et al., 2015, Molecular Therapy 23(1): 184-191; Han et al. 2018, Am J Cancer Res 8(1):106-119; Qin et al., 2017; Hematol & Oncol. 10:68; Jachimowicz et al. 2011, Molecular Cancer Therapeutics 10(6): 1036-1045; Schau et al. 2019, Scientific reports 9:3299; Han et al. 2017, Clin Cancer Res 23(13):3385-3395; Nejatollahi et al., 2013, Journal of Oncology Vol. 2013, Article ID 839831; Kugler et al, 2010 BJH Vol 150, 574-586.

### Costimulation

In some embodiments, the invention provides compositions and methods comprising a CAR-T cell population comprising a costimulatory polypeptide.

While CARs were first designed with a single signaling domain, for example, CD3ζ, also known as “first generation CARs” (see, e.g., Becker et al. (1989) Cell 58:911-921; Goverman et al. (1990) Cell 60:929-939; Gross et al. (1989) Proc Natl Acad Sci U.S.A. 86:10024-10028; Kuwana et al. (1987) Biochem Biophys Res Commun 149:960-968), clinical trials evaluating the feasibility of CAR immunotherapy showed limited clinical benefit (see, e.g., Till et al. (2012) Blood 119:3040-3050; Pule et al. (2008) Nat Med 14:1264-1270; Jensen et al. (2010) Biol Blood Marrow Transplant 16:1245-1256; Park et al. (2007) Mol Ther 15:825-833). The limited clinical benefit has been primarily attributed to the incomplete activation of T cells following tumor recognition, which leads to limited persistence and expansion of the cells in vivo (see, e.g., Ramos et al. (2011) Expert Opin Biol Ther 11:855-873).

To address this deficiency, CARs have been engineered to include another stimulating domain, often derived from the cytoplasmic portion of T cell costimulating molecules, including CD28, 4-1BB, OX40, ICOS and DAP10 (see, e.g., Carpenito et al. (2009) Proc Natl Acad Sci U.S.A. 106:3360-3365; Finney et al. (1998) J Immunol 161:2791-2797; Hombach et al. J Immunol 167:6123-6131; Maher et al. (2002) Nat Biotechnol 20:70-75; Imai et al. (2004) Leukemia 18:676-684; Wang et al. (2007) Hum Gene Ther 18:712-725; Zhao et al. (2009) J Immunol 183:5563-5574; Milone et al. (2009) Mol Ther 17:1453-1464; Yvon et al. (2009) Clin Cancer Res 15:5852-5860), which allow CAR-T cells to receive appropriate costimulation upon engagement of the target antigen. The most commonly used costimulating molecules include CD28 and 4-1BB, which, following tumor recognition, can initiate a signaling cascade resulting in NF-κB activation, which promotes both T cell proliferation and cell survival. Clinical trials conducted with anti-CD19 CARs having CD28 or 4-1BB signaling domains for the treatment of refractory acute lymphoblastic leukemia (ALL) have demonstrated significant T cell persistence, expansion and serial tumor killing following adoptive transfer (Kalos et al. (2011) Sci Transl Med 3:95ra73; Porter et al. (2011) N Engl J Med 365:725-733; Brentjens et al. (2013) Sci Transl Med 5:177ra38). Third generation CAR-T cells append CD28-modified CARs with additional signaling molecules from tumor necrosis factor (TNF)-family proteins, such as OX40 and 4-1BB (Finney H M, et al. J Immunol 172:104-13, 2004; Guedan S, et al., Blood, 2014).

In some embodiments, the compositions described herein containing DMSO and HSA comprise CAR-T cells.

In some embodiments, the compositions described herein containing DMSO and HSA comprise CAR-T cells comprising costimulatory polypeptides for enhancing and maintaining chimeric antigen receptor-expressing T cells.

The costimulatory polypeptide can be inducible or constitutively activated. The costimulatory polypeptide can comprise one or more costimulatory signaling regions such as CD27, ICOS, RANK, TRANCE, CD28, 4-1BB, OX40, DAP10, MyD88, or CD40 or, for example, the cytoplasmic regions thereof. The costimulatory polypeptide can comprise one or more suitable costimulatory signaling regions that activate the signaling pathways activated by CD27, ICOS, RANK, TRANCE, CD28, 4-1BB, OX40, DAP10, MyD88, or CD40. Costimulating polypeptides include any molecule or polypeptide that activates the NF-κB pathway, Akt pathway, and/or p38 pathway of tumor necrosis factor receptor (TNFR) family (i.e., CD40, RANK/TRANCE-R, OX40, 4-1BB) and CD28 family members (CD28, ICOS). More than one costimulating polypeptide or costimulating polypeptide cytoplasmic region may be expressed in the modified T cells discussed herein.

### Costimulation Provided by MyD88 and CD40

In some embodiments, the CAR T cell population formulated in the DMSO and HSA compositions described herein comprises a costimulatory polypeptide comprising one or more costimulatory signaling regions that activate the signaling pathways activated by CD27, ICOS, RANK, TRANCE, CD28, 4-1BB, OX40, DAP10, MyD88, or CD40

In some embodiments, the CAR T cell population formulated in the DMSO and HSA compositions described herein comprise a costimulatory polypeptide comprising one or more costimulatory signaling regions that activate the signaling pathways activated by MyD88, CD40 and/or MyD88-CD40 fusion chimeric polypeptide.

MyD88 is a universal adaptor molecule for TLRs and a critical signaling component of the innate immune system, triggering an alert for foreign invaders, priming immune cell recruitment and activation. MyD88 is a cytosolic adapter protein that plays a central role in the innate and adaptive immune response. This protein functions as an essential signal transducer in the interleukin-1 and Toll-like receptor signaling pathways. These pathways regulate that activation of numerous proinflammatory genes. The encoded protein consists of an N-terminal death domain and a C-terminal Toll-interleukin1 receptor domain. MyD88 TIR domain is able to heterodimerize with TLRs and homodimerize with other MyD88 proteins. This in turn results in recruitment and activation of IRAK family kinases through interaction of the death domains (DD) at the amino terminus of MyD88 and IRAK kinases which thereby initiates a signaling pathway that leads to activation of JNK, p38 MAPK (mitogen-activated protein kinase) and NF-κB, a transcription factor that induces expression of cytokine- and chemokine-encoding genes (as well as other genes). MyD88 acts acts via IRAK1, IRAK2, IRF7 and TRAF6, leading to NF-kappa-B activation, cytokine secretion and the inflammatory response. It also Activates IRF1 resulting in its rapid migration into the nucleus to mediate an efficient induction of IFN-beta, NOS2/INOS, and IL12A genes. MyD88-mediated signaling in intestinal epithelial cells is crucial for maintenance of gut homeostasis and controls the expression of the antimicrobial lectin REG3G in the small intestine. TLR signaling also upregulates expression of CD40, a member of the tumor necrosis factor receptor (TNFR) family, which interacts with CD40 ligand (CD154 or CD40L) on antigen-primed CD4+ T cells.

CD40 is an important part of the adaptive immune response, aiding to activate APCs through engagement with its cognate CD40L, in turn polarizing a stronger CTL response. The CD40/CD154 signaling system is an important component in T cell function and B cell—T cell interactions. CD40 signaling proceeds through formation of CD40 homodimers and interactions with TNFR-associated factors (TRAFs), carried out by recruitment of TRAFs to the cytoplasmic domain of CD40, which leads to T cell activation involving several secondary signals such as the NF-κB, JNK and AKT pathways.

Apart from survival and growth advantages, MyD88 or MyD88-CD40 fusion chimeric polypeptide-based costimulation may also provide additional functions to CAR-modified T cells. MyD88 signaling is critical for both Th1 and Th17 responses and acts via IL-1 to render CD4+ T cells refractory to regulatory T cell (Treg)-driven inhibition (see, e.g., Schenten et al. (2014) Immunity 40:78-90). In addition, CD40 signaling in CD8+ T cells via Ras, PI3K and protein kinase C, results in NF-κB-dependent induction of cytotoxic mediators granzyme and perforin that lyse CD4+CD25+ Treg cells (Martin et al. (2010) J Immunol 184:5510-5518). Thus, MyD88 and CD40 co-activation may render CAR-T cells resistant to the immunosuppressive effects of Treg cells, a function that could be critically important in the treatment of solid tumors and other types of cancers.

MyD88 and CD40 together in immune cells, including T cells, can act downstream on transcription factors to upregulate proinflammatory cytokines, Type I IFNs, and promote proliferation and survival. Along with signaling input from CD3z from a CAR, MyD88/CD40 makes for a potent and pleotropic costimulatory molecule. In some embodiments, the invention provides for CAR T cells comprising a costimulatory polypeptide comprising one or more costimulatory signaling regions that activate the signaling pathways activated by MyD88, CD40 and/or MyD88-CD40 fusion chimeric polypeptide. Examples of suitable costimulatory signaling regions include, but are not limited to, IRAK-4, IRAK-1, TRAF6, TRAF2, TRAF3, TRAFS, Act, JAK3, or any functional fragments thereof.

Non-limiting examples of chimeric polypeptides useful for inducing cell activation, and related methods for inducing CAR-T cell activation including, for example, expression constructs, methods for constructing vectors, and assays for activity or function, may also be found in the following patents and patent applications, each of which is incorporated by reference herein in its entirety for all purposes. US2016-0046700-A1; WO2015/123527; U.S. Pat. No. 7,404,950; WO2004/073641; U.S. Pat. No. 8,691,210; WO2008/049113; U.S. Pat. No. 9,315,559; WO2010/033949; US2011-0287038-A1; WO2011/130566; US2016-0175359-A1; WO2016/036746; WO2016/100241; US2017-0166877-A1; WO2017/106185; WO2018/208849, by Bayle et al., each of which is incorporated by reference herein in its entirety, including all text, tables and drawings, for all purposes.

### Safety Switches

Genetically-modified T cells formulated in the DMSO and HSA compositions described herein may express a safety switch, also known as an inducible suicide gene or suicide switch. This can be used to eradicate the T cells in vivo if desired e.g. if GVHD develops. In some examples, T cells that express a chimeric antigen receptor are provided to the patient that trigger an adverse event, such as off-target toxicity. In some therapeutic instances, a patient might experience a negative symptom during therapy using chimeric antigen receptor-modified cells. In some cases these therapies have led to side effects due, in part, to non-specific attacks on healthy tissue. In some examples, the therapeutic T cells may no longer be needed, or the therapy is intended for a specified amount of time, for example, the therapeutic T cells may work to decrease the tumor cell, or tumor size, and may no longer be needed. Therefore, in some embodiments are provided nucleic acids, cells, and methods wherein the modified T cell formulated in the DMSO and HSA compositions described herein also expresses an inducible Caspase-9 polypeptide. If there is a need, for example, to reduce the number of chimeric antigen receptor modified T cells, an inducible ligand may be administered to the patient, thereby inducing apoptosis of the modified T cells.

These switches respond to a trigger, such as a pharmacological agent, which is supplied when it is desired to eradicate the T cells, and which leads to cell death (e.g. by triggering necrosis or apoptosis). These agents can lead to expression of a toxic gene product, but a more rapid response can be obtained if the genetically-modified T cells already express a protein which is switched into a toxic form in response to the agent.

In some embodiments, a safety switch is based on a pro-apoptotic protein that can be triggered by administering a chemical inducer of dimerization to a subject. If the pro-apoptotic protein is fused to a polypeptide sequence which binds to the chemical inducer of dimerization, delivery of this chemical inducer can bring two pro-apoptotic proteins into proximity such that they trigger apoptosis. For instance, Caspase-9 can be fused to a modified human FK-binding protein which can be induced to dimerize in response to the pharmacological agent rimiducid (AP1903). The use of a safety switch based on a human pro-apoptotic protein, such as, for example, Caspase-9 minimizes the risk that cells expressing the switch will be recognized as foreign by a human subject's immune system. Delivery of rimiducid to a subject can therefore trigger apoptosis of T cells which express the caspase-9 switch.

Caspase-9 switches are described in Di Stasi et al. (2011) supra; see also Yagyu et al. (2015) Mol Ther 23(9):1475-85; Rossigloni et al. (2018) Cancer Gene Ther doi.org/10.1038/s41417-018-0034-1; Jones et al. (2014) Front Pharmacol doi.org/10.3389/fphar.2014.00254; U.S. Pat. Nos. 9,434,935; 9,913,882; 9,393,292; and patent application US2015/0328292. Suicide switches may also be based on Fas or on HSV thymidine kinase.

Examples of ligand inducers for the switches include, for example, those discussed in Kopytek, S. J., et al., Chemistry & Biology 7:313-321 (2000) and in Gestwicki, J. E., et al., Combinatorial Chem. & High Throughput Screening 10:667-675 (2007); Clackson T (2006) Chem Biol Drug Des 67:440-2; Clackson, T., in Chemical Biology: From Small Molecules to Systems Biology and Drug Design (Schreiber, s., et al., eds., Wiley, 2007).

The ligand binding regions incorporated in the safety switches may comprise the FKBP12v36 modified FKBP12 polypeptide, or other suitable FKBP12 variant polypeptides, including variant polypeptides that bind to AP1903, or other synthetic homodimerizers such as, for example, AP20187 or AP2015. Variants may include, for example, an FKBP region that has an amino acid substitution at position 36 selected from the group consisting of valine, leucine, isoleuceine and alanine (Clackson T, et al., Proc Natl Acad Sci USA. 1998, 95:10437-10442). AP1903, also known as rimiducid, (CAS Index Name: 2-Piperidinecarboxylic acid, 1-[(2S)-1-oxo-2-(3,4,5-trimethoxyphenyl)butyl]1,2-ethanediylbis[imino(2-oxo-2, 1-ethanediyl)oxy-3,1-phenylene[(1R)-3-(3,4-dimethoxyphenyl)propylidene]]ester, [2S-[1(R*),2R*[S*[S*[1(R*),2R*]]]]]-(9CI) CAS Registry Number: 195514-63-7; Molecular Formula: C78H98N4O20 Molecular Weight: 1411.65), is a synthetic molecule that has proven safe in healthy volunteers (Iuliucci J D, et al., J Clin Pharmacol. 2001, 41:870-879).

Provided in some embodiments are safety switches such as, for example, the safety switches discussed in Di Stasi et al. (2011) supra, which consists of the sequence of the human FK506-binding protein (FKBP12) (GenBank AH002 818) with an F36V mutation, connected through a SGGGS linker to a modified human caspase 9 (CASP9) which lacks its endogenous caspase activation and recruitment domain. The F36V mutation increases the binding affinity of FKBP12 to synthetic homodimerizers AP20187 and AP1903 (rimiducid).

The safety switch may comprise a modified Caspase-9 polypeptide having modified activity, such as, for example, reduced basal activity in the absence of the homodimerizer ligand. Modified Caspase-9 polypeptides are discussed in, for example, U.S. Pat. No. 9,913,882 and US-2015-0328292, supra, and may include, for example, amino acid substitutions at position 330 (e.g., D330E or D330A) or, for example, amino acid substitutions at position 450 (e.g., N405Q), or combinations thereof, including, for example, D330E-N405Q and D330A-N405Q.

An effective amount of a pharmaceutical composition, such as the dimerizing or multimerizing ligand presented herein, would be the amount that achieves this selected result of inducing apoptosis in the Caspase-9-expressing cells T cells, such that over 60%, 70%, 80%, 85%, 90%, 95%, or 97%, or that under 80%, 70%, 60%, 50%, 40%, 30%, 20%, or 10% of the therapeutic cells are killed. The term is also synonymous with “sufficient amount.” Any appropriate assay may be used to determine the percent of therapeutic cells that are killed. An assay may include the steps of obtaining a first sample from a subject before administration of the dimerizing or multimerizing ligand, and obtaining a second sample from the subject after administration of the dimerizing or multimerizing ligand, and comparing the number or concentration of therapeutic cells in the first and second samples to determine the percent of therapeutic cells that are killed. One can empirically determine the effective amount of a particular composition presented herein without necessitating undue experimentation.

Non-limiting examples of chimeric polypeptides useful for inducing cell death or apoptosis, and related methods for inducing cell death or apoptosis, including expression constructs, methods for constructing vectors, assays for activity or function, and multimerization of the chimeric polypeptides by contacting cells that express inducible chimeric polypeptides with a multimeric compound, or a pharmaceutically acceptable salt thereof, that binds to the multimerizing region of the chimeric polypeptides both ex vivo and in vivo, administration of expression vectors, cells, or multimeric compounds described herein, or pharmaceutically acceptable salts thereof, to subjects, and administration of multimeric compounds described herein, or pharmaceutically acceptable salts thereof, to subjects who have been administered cells that express the inducible chimeric polypeptides, may also be found in the following patents and patent applications, each of which is incorporated by reference herein in its entirety for all purposes. U.S. Pat. Nos. 9,089,520; 9,434,935; WO2014/16438; US2016-0151465-A1; WO2014/197638; US2015-0328292-A1; WO2015/134877; US2016-0166613-A1; WO2016/100236; US2016-0175359-A1 WO2016/100241; US2017-0166877-A1; WO2017/106185, each of which is incorporated by reference herein in its entirety, including all text, tables and drawings, for all purposes. Multimeric compounds described herein, or pharmaceutically acceptable salts thereof, may be used essentially as discussed in examples provided in these publications, and other examples provided herein.

### Methods of Treatment

The terms “patient” or “subject″” are interchangeable, and, as used herein include, but are not limited to, an organism or animal; a mammal, including, e.g., a human, non-human primate (e.g., monkey), mouse, pig, cow, goat, rabbit, rat, guinea pig, hamster, horse, monkey, sheep, or other non-human mammal; a non-mammal, including, e.g., a non-mammalian vertebrate, such as a bird (e.g., a chicken or duck) or a fish, and a non-mammalian invertebrate. The subject may be, for example, human, for example, a patient suffering from an infectious disease, and/or a subject that is immunocompromised, or is suffering from a hyperproliferative disease.

Modified cell populations provided herein may be used in methods for treating human subjects in need thereof, and may be used to prepare medicaments for treating such subjects. The cells will usually be delivered to the recipient subject by infusion (typically intravenous infusion). A typical dose of T cells for the subject is between 10⁴-10⁹ cells/kg, for example 1×10⁴, 5×10⁴, 1×10⁵, 2×10⁵, 3×10⁵, 4×10⁵, 5×10⁵, 6×10⁵, 7×10⁵, 8×10⁵, 9×10⁵, 1×10⁶, 2×10⁶, 3×10⁶, 4×10⁶, 5×10⁶, 6×10⁶, 7×10⁶, 8×10⁶, 9×10⁶, 1×10⁷, 2×10⁷, 3×10⁷, 4×10⁷, 5×10⁷, 6×10⁷, 7×10⁷, 8×10⁷, 9×10⁷, 1×10⁸, 2×10⁸, 3×10⁸, 4×10⁸, 5×10⁸, 6×10⁸, 7×10⁸, 8×10⁸, 9×10⁸, or 1×10⁹ modified cells, or cells from the modified cell population, per kg subject body weight are administered to the subject. In some embodiments, the dosage is based on a desired fixed dose of total cells and a desired ratio, and/or based on a desired fixed dose of one or more, e.g., each, of the individual sub-types or sub-populations.

In general, from 20-200 million T cells may be infused into a subject e.g. from 30-120 million T cells.

The number of cells delivered may be the same for all patients in a particular category (e.g. all pediatric patients, or all adult patients), or it may vary according to body weight e.g. about 1×10⁶ cells/kg for children or about 3×10⁶ cells/kg for adults. The volume which is administered to a patient may therefore vary depending on the concentration of cells in the composition, and optionally according to the patient's body weight, to ensure that the desired number of cells is received.

The recipient may undergo myeloablative conditioning prior to receiving the modified cell population comprising genetically-modified T cells. Thus, the recipient's own α/β T cells (and B cells) can be depleted prior to receiving the genetically-modified T cells. Similarly, haematopoietic (stem) cells which are administered to a recipient may be depleted for α/β cells. In contrast, genetically-modified donor T cells administered to the recipient are generally not depleted for α/β cells.

The recipient can be a child e.g. a child aged from 0-16 years old, or from 0-10 years old. In some embodiments, the recipient is an adult.

Subjects receiving the T cells may also receive other tissue from an allogeneic donor e.g. they can receive haematopoietic cells and/or haematopoietic stem cells (e.g. CD34+ cells). This allograft tissue and the genetically-modified T cells are ideally derived from the same donor, such that they will be genetically matched. In some embodiments, the donor and the recipient are a matched unrelated donor, or a suitable family member. For instance, the donor may be the recipient's parent or child. Where a subject is identified as being in need of genetically-modified T cells, therefore, a suitable donor can be identified as a T cell donor.

Where modified cell populations provided herein (for example, modified cell populations comprising modified T cells) are used in conjunction with haematopoietic cells and/or haematopoietic stem cells, the modified cell populations may, in some examples, be administered at a later timepoint e.g. between 7-100 days later.

In some embodiments, where the T cell population comprise a costimulatory polypeptide, the costimulatory polypeptide can be triggered e.g. by administering an effective amount of an inducible ligand (e.g., rimiducid or a rapalog) to the recipient. In some embodiments the costimulatory polypeptide comprises one or more costimulatory signaling regions that activate the signaling pathways activated by MyD88, CD40 and/or MyD88-CD40 fusion chimeric polypeptide.

If the recipient develops complications after receiving the genetically-modified T cells (e.g. they develop GVHD) then the suicide switch can be triggered e.g. by administering an inducible ligand (e.g., rimiducid or a rapalog) to the recipient. The minimum dose of the inducible ligand (e.g., rimiducid or a rapalog) required to eliminate the modified cells, where the modified cells comprise an inducible chimeric pro-apoptotic polypeptide, will depend on the number of genetically-modified T cells which are present in the recipient. Doses above this minimum can be administered but, in accordance with normal pharmaceutical principles, excessive dosing should be avoided. In some embodiments, the suicide switch can be triggered with rimiducid, e.g., a dose of 0.4 mg/kg can eliminate cells which were infused at a dose of 1.5×107 cells/kg. In general terms, a rimiducid dose between 0.1-5 mg/kg is administered, and usually 0.1-2 mg/kg or 0.1 lmg/kg will suffice, and, in some embodiments, the dose is 0.4 mg/kg. A series of multiple doses of rimiducid can be administered e.g. if it is found that a first dose does not eliminate all genetically-modified T cells then a second dose can be administered, etc.

In some embodiments, a first dose of the inducing ligand (e.g. rimiducid) is administered which kills the most sensitive cells, and then a second dose (which is higher than the first dose) is administered which kills cells which are less sensitive. Further doses (escalating where necessary) can be administered if required.

The present methods also encompass methods of treatment or prevention of a disease caused by pathogenic microorganisms and/or a hyperproliferative disease.

Diseases that may be treated or prevented include diseases caused by viruses, bacteria, yeast, parasites, protozoa, cancer cells and the like. The pharmaceutical composition may be used as a generalized immune enhancer (T cell activating composition or system) and as such has utility in treating diseases. Exemplary diseases that can be treated and/or prevented include, but are not limited, to infections of viral etiology such as HIV, influenza, Herpes, viral hepatitis, Epstein Bar, polio, viral encephalitis, measles, chicken pox, Papilloma virus etc.; or infections of bacterial etiology such as pneumonia, tuberculosis, syphilis, etc.; or infections of parasitic etiology such as malaria, trypanosomiasis, leishmaniasis, trichomoniasis, amoebiasis, etc.

Preneoplastic or hyperplastic states which may be treated or prevented using the pharmaceutical composition (transduced T cells, expression vector, expression construct, etc.) include but are not limited to preneoplastic or hyperplastic states such as colon polyps, Crohn's disease, ulcerative colitis, breast lesions and the like.

Cancers, including solid tumors, which may be treated using the composition include, but are not limited to primary or metastatic melanoma, adenocarcinoma, squamous cell carcinoma, adenosquamous cell carcinoma, thymoma, lymphoma, sarcoma, lung cancer, liver cancer, non-Hodgkin's lymphoma, Hodgkin's lymphoma, leukemias, uterine cancer, breast cancer, prostate cancer, ovarian cancer, pancreatic cancer, colon cancer, multiple myeloma, neuroblastoma, NPC, bladder cancer, cervical cancer and the like.

Solid tumors from any tissue or organ may be treated using the present methods, including, for example, for example, solid tumors present in, for example, lungs, bone, liver, prostate, or brain, and also, for example, in breast, ovary, bowel, testes, colon, pancreas, kidney, bladder, neuroendocrine system, soft tissue, boney mass, and lymphatic system. Other solid tumors that may be treated include, for example, glioblastoma, and malignant myeloma.

The recipient may have a hematological cancer (such as a treatment-refractory hematological cancer) or an inherited blood disorder. For instance, the recipient may have acute lymphoblastic leukemia (ALL), acute myeloid leukemia (AML), severe combined immune-deficiency (SCID), Wiskott-Aldrich syndrome (WA), Fanconi Anemia, chronic myelogenous leukemia (CML), non-Hodgkin lymphoma (NHL), Hodgkin lymphoma (HL), or multiple myeloma.

The term “cancer” as used herein is defined as a hyperproliferation of cells whose unique trait—loss of normal controls—results in unregulated growth, lack of differentiation, local tissue invasion, and metastasis. Examples include but are not limited to, melanoma, non-small cell lung, small-cell lung, lung, hepatocarcinoma, leukemia, retinoblastoma, astrocytoma, glioblastoma, gum, tongue, neuroblastoma, head, neck, breast, pancreatic, prostate, renal, bone, testicular, ovarian, mesothelioma, cervical, gastrointestinal, lymphoma, brain, colon, sarcoma or bladder.

The term “hyperproliferative disease” is defined as a disease that results from a hyperproliferation of cells. Other hyperproliferative diseases, including solid tumors, that may be treated using the T cell and other therapeutic cell activation system presented herein include, but are not limited to rheumatoid arthritis, inflammatory bowel disease, osteoarthritis, leiomyomas, adenomas, lipomas, hemangiomas, fibromas, vascular occlusion, restenosis, atherosclerosis, pre-neoplastic lesions (such as adenomatous hyperplasia and prostatic intraepithelial neoplasia), carcinoma in situ, oral hairy leukoplakia, or psoriasis.

As used herein, the terms “treatment”, “treat”, “treated”, or “treating” refer to prophylaxis and/or therapy. When used with respect to a solid tumor, such as a cancerous solid tumor, for example, the term refers to prevention by prophylactic treatment, which increases the subject's resistance to solid tumors or cancer. In some examples, the subject may be treated to prevent cancer, where the cancer is familial, or is genetically associated. When used with respect to an infectious disease, for example, the term refers to a prophylactic treatment which increases the resistance of a subject to infection with a pathogen or, in other words, decreases the likelihood that the subject will become infected with the pathogen or will show signs of illness attributable to the infection, as well as a treatment after the subject has become infected in order to fight the infection, for example, reduce or eliminate the infection or prevent it from becoming worse.

The methods provided herein may be used, for example, to treat a disease, disorder, or condition wherein there is an elevated expression of a tumor antigen.

The administration of the pharmaceutical composition may be for either “prophylactic” or “therapeutic” purpose. When provided prophylactically, the pharmaceutical composition is provided in advance of any symptom. The prophylactic administration of modified cell populations serves to prevent or ameliorate any subsequent infection or disease. When provided therapeutically, the modified cell population is provided at or after the onset of a symptom of infection or disease. Thus the compositions presented herein may be provided either prior to the anticipated exposure to a disease-causing agent or disease state or after the initiation of the infection or disease. Thus provided herein are methods for prophylactic treatment of solid tumors such as those found in cancer, or for example, but not limited to, prostate cancer, using the modified cell populations discussed herein. For example, methods are provided of prophylactically preventing or reducing the size of a tumor in a subject comprising administering a the modified cell populations discussed herein, whereby the modified cell population is administered in an amount effect to prevent or reduce the size of a tumor in a subject.

An effective amount of the pharmaceutical composition described herein would be the amount that achieves this selected result of enhancing the immune response, and such an amount could be determined. For example, an effective amount of for treating an immune system deficiency could be that amount necessary to cause activation of the immune system, resulting in the development of an antigen specific immune response upon exposure to antigen. The term is also synonymous with “sufficient amount.” In other examples, an effective amount could be that amount necessary for reducing tumor size or the number of tumors, or for reducing the growth rate of tumors, or the rate of proliferation of tumors. In other examples, an effective amount could be that amount necessary for reducing the amount or concentration of target antigen in a subject, measured by comparing the amount or concentration of target antigen in samples obtained before, during, and/or after administration of the modified cell populations provided herein.

The effective amount for any particular application can vary depending on such factors as the disease or condition being treated, the particular composition being administered, the size of the subject, and/or the severity of the disease or condition. Thus, for example, in one embodiment, the transduced T cells or other cells are administered to a subject in an amount effective to, for example, induce an immune response, or, for example, to reduce the size of a tumor or reduce the amount of tumor vasculature.

In some embodiments, multiple doses of modified cells are administered to the subject, with an escalation of dosage levels among the multiple doses. In some embodiments, the escalation of dosage levels increases the level of CAR-T cell activity, and therefore increases the therapeutic effect, such as, for example, the reduction in the amount or concentration of target cells, such as, for example, tumor cells.

In some embodiments, personalized treatment is provided wherein the stage or level of the disease or condition is determined before administration of the modified cells, before the administration of an additional dose of the modified cells, or in determining method and dosage involved in the administration of the modified cells. These methods may be used in any of the methods of the present application. Where these methods of assessing the patient before administering the modified cells are discussed in the context of, for example, the treatment of a subject with a solid tumor, it is understood that these methods may be similarly applied to the treatment of other conditions and diseases. Thus, for example, in some embodiments of the present application, the method comprises administering the modified cells of the present application to a subject, and further comprises determining the appropriate dose of modified cells to achieve the effective level of reduction of tumor size. The amount of cells may be determined, for example, based on the subject's clinical condition, weight, and/or gender or other relevant physical characteristic. By controlling the amount of modified cells administered to the subject, the likelihood of adverse events such as, for example, a cytokine storm may be reduced.

The term “dosage” is meant to include both the amount of the dose and the frequency of administration, such as, for example, the timing of the next dose. The term “dosage level” refers to the amount of the cell population administered in relation to the body weight of the subject.

In some examples, the term dosage may refer to the dosage of the ligand inducer. For example, to induce the chimeric Caspase-9 polypeptide or the chimeric costimulatory polypeptide, the term “dosage level” refers to the amount of the multimeric ligand administered in relation to the body weight of the subject. Thus increasing the dosage level would mean increasing the amount of the ligand administered relative to the subject's weight. In addition, increasing the concentration of the dose administered, such as, for example, when the multimeric ligand is administered using a continuous infusion pump would mean that the concentration administered (and thus the amount administered) per minute, or second, is increased.

Methods as presented herein include without limitation the delivery of an effective amount of the DMSO and HSA compositions comprising the cell populations described herein. An “effective amount” of the cell population, generally, is defined as that amount sufficient to detectably and repeatedly to achieve the stated desired result, for example, to ameliorate, reduce, minimize or limit the extent of the disease or its symptoms. Other more rigorous definitions may apply, including elimination, eradication or cure of disease. In some embodiments there may be a step of monitoring the biomarkers, or other disease symptoms such as tumor size or tumor antigen expression, to evaluate the effectiveness of treatment and to control toxicity.

If needed, the method may further include additional leukaphereses to obtain more cells to be used in treatment.

### Combination Therapies

In order to increase the effectiveness of the compositions presented herein, it may be desirable to combine these compositions and methods with an agent effective in the treatment of the disease.

In certain embodiments, anti-cancer agents may be used in combination with the present methods. An “anti-cancer” agent is capable of negatively affecting cancer in a subject, for example, by killing one or more cancer cells, inducing apoptosis in one or more cancer cells, reducing the growth rate of one or more cancer cells, reducing the incidence or number of metastases, reducing a tumor's size, inhibiting a tumor's growth, reducing the blood supply to a tumor or one or more cancer cells, promoting an immune response against one or more cancer cells or a tumor, preventing or inhibiting the progression of a cancer, or increasing the lifespan of a subject with a cancer. Anti-cancer agents include, for example, chemotherapy agents (chemotherapy), radiotherapy agents (radiotherapy), a surgical procedure (surgery), immune therapy agents (immunotherapy), genetic therapy agents (gene therapy), hormonal therapy, other biological agents (biotherapy) and/or alternative therapies.

In some embodiments antibiotics can be used in combination with the pharmaceutical composition to treat and/or prevent an infectious disease. Such antibiotics include, but are not limited to, amikacin, aminoglycosides (e.g., gentamycin), amoxicillin, amphotericin B, ampicillin, antimonials, atovaquone sodium stibogluconate, azithromycin, capreomycin, cefotaxime, cefoxitin, ceftriaxone, chloramphenicol, clarithromycin, clindamycin, clofazimine, cycloserine, dapsone, doxycycline, ethambutol, ethionamide, fluconazole, fluoroquinolones, isoniazid, itraconazole, kanamycin, ketoconazole, minocycline, ofloxacin), para-aminosalicylic acid, pentamidine, polymixin definsins, prothionamide, pyrazinamide, pyrimethamine sulfadiazine, quinolones (e.g., ciprofloxacin), rifabutin, rifampin, sparfloxacin, streptomycin, sulfonamides, tetracyclines, thiacetazone, trimethaprim-sulfamethoxazole, viomycin or combinations thereof.

More generally, such an agent would be provided in a combined amount with the pharmaceutical compositions effective to kill or inhibit proliferation of a cancer cell and/or microorganism. This process may involve contacting the cell(s) with an agent(s) and the pharmaceutical composition at the same time or within a period of time wherein separate administration of the pharmaceutical composition and an agent to a cell, tissue or organism produces a desired therapeutic benefit. This may be achieved by contacting the cell, tissue or organism with a single composition or pharmacological formulation that includes both the pharmaceutical composition and one or more agents, or by contacting the cell with two or more distinct compositions or formulations, wherein one composition includes the pharmaceutical composition and the other includes one or more agents.

The terms “contacted” and “exposed,” when applied to a cell, tissue or organism, are used herein to describe the process by which the pharmaceutical composition and/or another agent, such as for example a chemotherapeutic or radiotherapeutic agent, are delivered to a target cell, tissue or organism or are placed in direct juxtaposition with the target cell, tissue or organism. To achieve cell killing or stasis, the pharmaceutical composition and/or additional agent(s) are delivered to one or more cells in a combined amount effective to kill the cell(s) or prevent them from dividing. In some embodiments, the chemotherapeutic agent is selected from the group consisting of carboplatin, estramustine phosphate (Emcyt), and thalidomide. In some embodiments, the chemotherapeutic agent is a taxane. The taxane may be, for example, selected from the group consisting of docetaxel (Taxotere), paclitaxel, and cabazitaxel. In some embodiments, the taxane is docetaxel. In some embodiments, the chemotherapeutic agent is administered at the same time or within one week after the administration of the modified cell or nucleic acid. In other embodiments, the chemotherapeutic agent is administered from 1 to 4 weeks or from 1 week to 1 month, 1 week to 2 months, 1 week to 3 months, 1 week to 6 months, 1 week to 9 months, or 1 week to 12 months after the administration of the modified cell or nucleic acid. In some embodiments, the chemotherapeutic agent is administered at least 1 month before administering the cell or nucleic acid.

The administration of the pharmaceutical composition may precede, be concurrent with and/or follow the other agent(s) by intervals ranging from minutes to weeks. In embodiments where the pharmaceutical composition and other agent(s) are applied separately to a cell, tissue or organism, one would generally ensure that a significant period of time did not expire between the times of each delivery, such that the pharmaceutical composition and agent(s) would still be able to exert an advantageously combined effect on the cell, tissue or organism. For example, in such instances, it is contemplated that one may contact the cell, tissue or organism with two, three, four or more modalities substantially simultaneously (i.e., within less than about a minute) with the pharmaceutical composition. In other embodiments, one or more agents may be administered within from substantially simultaneously, about 1 minute, to about 24 hours to about 7 days to about 1 to about 8 weeks or more, and any range derivable therein, prior to and/or after administering the expression vector. Yet further, various combination regimens of the pharmaceutical composition presented herein and one or more agents may be employed.

In some embodiments, the chemotherapeutic agent may be a lymphodepleting chemotherapeutic. In other examples, the chemotherapeutic agent may be Taxotere (docetaxel), or another taxane, such as, for example, cabazitaxel. The chemotherapeutic may be administered before, during, or after treatment with the cells and inducer. For example, the chemotherapeutic may be administered about 1 year, 11, 10, 9, 8, 7, 6, 5, or 4 months, or 18, 17, 16, 15, 14, 13, 12, 11, 10, 9, 8, 7, 6, 5, 4, 3, 2, weeks or 1 week prior to administering the first dose of activated nucleic acid. Or, for example, the chemotherapeutic may be administered about 1 week or 2, 3, 4, 5, 6, 7, 8, 9, 10, 11, 12, 13, 14, 15, 16, 17, or 18 weeks or 4, 5, 6, 7, 8, 9, 10, or 11 months or 1 year after administering the first dose of cells or inducer.

Administration of a chemotherapeutic agent may comprise the administration of more than one chemotherapeutic agent. For example, cisplatin may be administered in addition to Taxotere or other taxane, such as, for example, cabazitaxel.

In some embodiments, the recipient may undergo myeloablative conditioning prior to receiving the compositions comprising the cell populations described herein. Thus, the recipient's own α/β T cells (and B cells) can be depleted prior to receiving compositions comprising the cell populations described herein. In some embodiments a lymphodepleting chemotherapy regimen is administered before the infusion of the compositions comprising the cell populations described herein. In some embodiments, lymphodepleting chemotherapy regimen comprises cyclophosphamide 500 mg/m² intravenously and fludarabine 30 mg/m² intravenously was administering on the fifth, fourth, and third day before infusion, unless the combination is not tolerated. If it is determined that combination-based lymphodepletion presented an unfavorable safety risk to a subject, then lymphodepletion can be done with cyclophosphamide alone.

### General

The practice of the present invention will employ, unless otherwise indicated, conventional methods of chemistry, biochemistry, molecular biology, immunology and pharmacology, within the skill of the art. Such techniques are explained fully in the literature. See, e.g., Methods In Enzymology (S. Colowick and N. Kaplan, eds., Academic Press, Inc.), Handbook of Experimental Immunology, Vols. I-IV (D. M. Weir and C. C. Blackwell, eds, Blackwell Scientific Publications), Advanced Methods in Cellular Immunology (Fernandez-Botan & Větvička, 1st ed. 2000), Short Protocols in Immunology (Coligan, 2005), Green & Sambrook (2012) Molecular Cloning: A Laboratory Manual, 4th edition (Cold Spring Harbor Press), Ausubel et al. (eds) Short protocols in molecular biology, 5th edition (Current Protocols), Molecular Biology Techniques: An Intensive Laboratory Course, (Ream et al., eds., 1998, Academic Press), etc.

The term “comprising” encompasses “including” as well as “consisting” e.g. a composition “comprising” X may consist exclusively of X or may include something additional e.g. X+Y.

The term “about” in relation to a numerical value x is optional and means, for example, x±10%.

The word “substantially” does not exclude “completely” e.g. a composition which is “substantially free” from Y may be completely free from Y. Where necessary, the word “substantially” may be omitted from the definition of the invention.

The term “between” with reference to two values includes those two values e.g. the range “between” 10 mg and 20 mg encompasses inter alia 10, 15, and 20 mg.

Unless specifically stated, a method comprising a step of mixing two or more components does not require any specific order of mixing. Thus components can be mixed in any order. Where there are three components then two components can be combined with each other, and then the combination may be combined with the third component, etc.

The various steps of methods may be carried out at the same or different times, in the same or different geographical locations (e.g. countries) and by the same or different people or entities.

### NUMBERED EMBODIMENTS

1. A composition comprising T cells and serum albumin, wherein the composition comprises <2.5% (v/v) serum albumin.

2. The composition of embodiment 1, comprising <2.2% (v/v), <2% (v/v), <1% (v/v), or about 0.5% (v/v) serum albumin.

3. The composition of embodiment 1 or embodiment 2, also including dimethyl sulfoxide (DMSO).

4. The composition of embodiment 3, wherein DMSO is present at a 3-fold to 30 fold excess (v/v) compared to the serum albumin.

5. The composition of embodiment 4, wherein the excess is from 3 fold to 15-fold, from 6-fold to 12-fold, from 8-fold to 11-fold, or about 10 fold.

6. A composition comprising T cells, serum albumin, and DMSO, wherein the concentration of serum albumin (v/v) is from 0.1-2% and the concentration of DMSO (v/v) is 3-fold to 30-fold higher than the concentration of serum albumin.

7. The composition of embodiment 6, wherein the serum albumin concentration is <1% (v/v), and the DMSO concentration is from 8 fold to 11 fold higher than the serum albumin concentration.

8. The composition of embodiment 6 or embodiment 7, wherein concentration of DMSO is <10% (v/v).

9. The composition of embodiment 8, wherein the concentration of DMSO is from 2-8% (v/v), from 4-7% (v/v), from 5-6% (v/v), or about 5% (v/v).

10. A composition comprising T cells and DMSO, wherein the composition comprises <5% (v/v) DMSO.

11. A composition comprising T cells, serum albumin, and DMSO, wherein the concentration of serum albumin (v/v) is 0.1-1% and the concentration of DMSO is 5-7% (v/v).

12. The composition of embodiment 11, wherein the concentration of serum albumin (v/v) is between 0.2-0.8%, between 0.3-0.7%, between 0.4-0.6%, or about 0.5%.

13. The composition of any one of embodiments 1 to 12, having between 1×10⁶ and 40×10⁶ T cells/mL.

14. The composition of any one of embodiments 1 to 13, in cryopreserved form.

15. A composition comprising T cells and serum albumin, wherein the composition comprises <1% (v/v) serum albumin.

16. The composition of embodiment 15, comprising <0.6% (v/v) serum albumin or about 0.15% (v/v) serum albumin.

17. The composition of embodiment 15 or embodiment 16, also including DMSO.

18. The composition of embodiment 17, wherein DMSO is present at a 3-fold to 30 fold excess (v/v) compared to the serum albumin.

19. The composition of embodiment 18, wherein the excess is from 3 fold to 15-fold, from 6-fold to 12-fold, from 8-fold to 11-fold, or about 10 fold.

20. A composition comprising T cells, serum albumin, and DMSO, wherein the concentration of serum albumin (v/v) is from 0.03-0.6% and the concentration of DMSO (v/v) is 3-fold to 30-fold higher than the concentration of serum albumin.

21. The composition of embodiment 20, wherein the serum albumin concentration is <0.3% (v/v), and the DMSO concentration is from 8 fold to 11 fold higher than the serum albumin concentration.

22. The composition of embodiment 20 or embodiment 21, wherein concentration of DMSO is <3% (v/v).

23. The composition of embodiment 8, wherein the concentration of DMSO is from 0.6-2.4% (v/v), from 0.8-2.2% (v/v), from 1.5-2% (v/v), or about 1.8% (v/v).

24. A composition comprising T cells and DMSO, wherein the composition comprises <0.15% (v/v) DMSO.

25. A composition comprising T cells, serum albumin, and DMSO, wherein the concentration of serum albumin is 0.03-0.3% (v/v) and the DMSO concentration is 0.15-0.20% (v/v).

26. The composition of any one of embodiments 15 to 25, having between 0.3×10⁶ and 12×10⁶ T cells/mL.

27. The composition of any preceding embodiment, wherein the T cells are genetically modified T cells, such as CAR-T cells.

28. The composition of any preceding embodiment, wherein the T cells are human T cells and the serum albumin is human serum albumin.

29. A process for preparing the composition of any one of embodiments 15 to 28, comprising the step of diluting a composition of any one of embodiments 1 to 14.

30. The process of embodiment 29, wherein dilution is performed with an electrolyte solution.

31. The process of embodiment 30, wherein the electrolyte solution is normal saline.

32. The process of embodiment 30, wherein the electrolyte solution is a sterile, nonpyrogenic, isotonic solution with a pH of 6.5-8.0 that has about 5.26 g/L NaCl, about 5.02 g/L sodium gluconate, about 3.68 g/L sodium acetate trihydrate, about 0.37 g/L KCl, and about 0.3 g/L MgCl₂. 6H₂O.

33. A method of treating a subject, comprising a step of administering a composition of any one of embodiments 1 to 28 to a patient in need thereof.

34. A container comprising a suspension of T cells, serum albumin and dimethyl sulfoxide (DMSO), wherein the composition comprises <2.5% (v/v) serum albumin, and wherein DMSO is present at a ≥5-fold excess (v/v) compared to the serum albumin, and wherein the suspension is to be administered to a patient.

35. The container of embodiment 34, wherein the suspension comprises <2.2% (v/v), <2% (v/v), <1% (v/v), or about 0.5% (v/v) serum albumin.

36. The container of embodiment 34 or embodiment 35, wherein DMSO is present at a 5-fold to 30-fold excess (v/v) compared to the serum albumin.

37. The container of embodiment 36, wherein the excess is from 5-fold to 15-fold, from 6-fold to 12-fold, from 8-fold to 11-fold, or about 10-fold.

38. A container comprising a suspension of T cells, serum albumin, and DMSO, wherein the concentration of serum albumin (v/v) is from 0.1-1% and the concentration of DMSO (v/v) is 3-fold to 30-fold higher than the concentration of serum albumin, and wherein the suspension is to be administered to a patient.

39. The container of embodiment 38, wherein the serum albumin concentration is <1% (v/v), and the DMSO concentration is from 8-fold to 11-fold higher than the serum albumin concentration.

40. The container of embodiment 38 or embodiment 39, wherein concentration of DMSO is <10% (v/v).

41. The container of embodiment 40, wherein the concentration of DMSO is from 2-8% (v/v), from 4-7% (v/v), from 5-6% (v/v), or about 5% (v/v).

42. A container comprising a suspension of T cells, serum albumin, and DMSO, wherein the concentration of serum albumin (v/v) is 0.1-1% and the concentration of DMSO is 5-7% (v/v), and wherein the suspension is to be administered to a patient.

43. The container of embodiment 42, wherein the concentration of serum albumin (v/v) is between 0.2-0.8%, between 0.3-0.7%, between 0.4-0.6%, or about 0.5%.

44. The container of any one of embodiments 34 to 43, having between 1×10⁶ and 40×10⁶ T cells/mL.

45. The composition of any one of embodiments 34 to 44, in cryopreserved form.

46. A method of treating a subject, comprising a step of administering to a patient in need thereof the composition of any one of embodiments 15 to 28 by intravenous infusion.

47. The method of embodiment 46, wherein the intravenous infusion time is between 15 and 120 minutes.

48. The method of embodiment 46, wherein the intravenous infusion time is up to 30 minutes.

49. The method of any one of embodiments 44-46, wherein the infusion volume is between 50 and 100 mL.

50. The method of any one of embodiments 46-49, wherein the infusion volume is about 50 mL.

51. The composition, method, process, or container of any preceding embodiment, wherein the T cells comprise a polynucleotide that encodes a chimeric signaling polypeptide, wherein the chimeric signaling polypeptide comprises:
(i) a costimulatory polypeptide cytoplasmic signaling region;
(ii) a truncated MyD88 polypeptide region lacking the TIR domain;
(iii) a truncated MyD88 polypeptide region lacking the TIR domain and a costimulatory polypeptide cytoplasmic signaling region; or
(iv) (iv) a truncated MyD88 polypeptide region lacking the TIR domain and a CD40 cytoplasmic polypeptide region lacking the CD40 extracellular domain.

52. The composition, method, process, or container of embodiment 48, wherein costimulatory polypeptide cytoplasmic signaling region is a signaling region that activates the signaling pathways activated by MyD88, CD40 and/or MyD88-CD40 fusion chimeric polypeptide.

53. The composition, method, process, or container of any of the preceding embodiment, wherein the T cells comprises a polynucleotide encoding an inducible chimeric pro-apoptotic polypeptide.

54. A kit comprising:
(a) a container comprising an interior space; and
(b) a composition contained within the interior space, wherein the composition comprises:
   (i) T cells in an amount between about 1×10⁶ and about 40×10⁶ (e.g., about 5×10⁶) cells/ml;
   (ii) serum albumin in a concentration of about 0.1%-0.6% v:v;
   (iii) DMSO in a concentration of about 5%-7% v:v; and
   (iv) a pharmaceutically acceptable carrier for human injection.

55. The kit of embodiment 54, wherein the container comprises a bag, e.g., a cryopreservation bag.

56. The kit of embodiment 55, wherein the container further comprises a bag spike in fluidic communication with the interior space.

57. The kit of embodiment 54, wherein the T cells are genetically engineered.

58. The kit of embodiment 54, wherein the serum albumin is human serum albumin.

59. The kit of embodiment 54, wherein the serum albumin is present in concentration of about 0.5% v:v.

60. The kit of embodiment 54, wherein DMSO is present in a concentration of about 5% v:v.

61. The kit of embodiment 54, wherein the pharmaceutically acceptable carrier comprises sodium chloride, sodium gluconate sodium, acetate trihydrate, potassium chloride, and magnesium chloride.

62. The kit of embodiment 54, wherein the pharmaceutically acceptable carrier comprises Pharma Lyte or Plasma Lyte.

63. The kit of embodiment 54, wherein the composition has a volume of about 15 ml.

64. A kit comprising:
(a) a container comprising an interior space; and
(b) a composition contained within the interior space, wherein the composition comprises:
   (i) T cells in an amount between about 15×10⁶ and about 600×10⁶ (e.g., about 75×10⁶) cells;
   (ii) serum albumin in an amount between about 0.03% and about 1.8% serum albumin, e.g., about 1.5% v:v;
   (iii) DMSO in a concentration of about 1.5%-2.1 v:v (e.g., about 1.5% v:v); and
   (iv) a pharmaceutically acceptable carrier for human injection.

65. The kit of embodiment 63, wherein the container comprises a bag, optionally including a tube comprising a bag spike, wherein the tube is in fluidic communication with the interior space.

66. The kit of embodiment 63, wherein the composition has a volume of about 50 ml.

67. A composition as described herein for use in treating a disease, e.g., cancer.

68. A use of a composition as described herein for treating a disease, e.g., cancer.

69. A use of a composition as described herein in the preparation of a medicament for treating a disease, e.g., cancer.

### EXAMPLES

### Example 1: Preparation of Genetically Modified T Cells

Cells were obtained essentially as follows:
Cells are obtained from a sample of a donor's peripheral blood by leukapheresis, to enrich for white blood cells. Mononuclear cells are enriched by density gradient separation using a CliniMACS Prodigy™ apparatus (Miltenyi Biotec), and are cryopreserved for storage at −130° C.
When required, they are thawed in a dry bath and then (day 1) cultured on the Prodigy™ apparatus in a culture bag at 37° C. and 5% CO₂. The cells are seeded into the culture medium at 1-3×10⁶ cells/mL. The medium is a serum-free medium which supports T cells (containing transferrin, albumin, and insulin), supplemented with 100 U/mL IL-2, 0.2 μg/mL anti-CD3 mAb (OKT3 from Miltenyi Biotec), and 0.5 μg/mL anti-CD28 mAb. These conditions activate the T cells.
On day 3 the culture is fed with fresh 100 U/mL IL-2 and 33% additional fresh medium. On day 5 the cells are transferred to a RetroNectin™-coated AC bag and are transduced with the Gal-V retroviral vector encoding the iCasp9 suicide switch disclosed by Tey et al. (2007) Biol Blood Marrow Transpl 13:913-24 and Di Stasi et al. (2011) N Engl J Med 365:1673-83. IL-2 is added at 200 U/mL.
On day 6 the cells are processed to remove remaining retroviral particles (supernatant), and are re-suspended in medium at 10⁶ cells/mL with 200 U/mL IL-2. Culture continues at 37° C., 5% CO₂.
On day 8 transduced cells are selected by MACS on the basis of CD19 expression, again using the Prodigy™ system. The CD19+ cells are cultured in a bag, as before, with further IL-2 (200 IU/mL) at 10⁶ cells/mL.
On day 9 the cells are harvested, washed with Plasma-Lyte A (Rizoli (2011) J Trauma 70(5 Suppl) S17-8) in a centrifuge, and resuspended in cryopreservation medium for storage at −130° C. until they are needed by an allogeneic recipient.
If, however, cell numbers were not adequate at day 9 (e.g. too few cells for the intended treatment) then culture can be continued up to day 15 prior to harvest, as described in the previous point.

The resulting cells are suitable for re-introduction to the donor, and can provide virus and tumor-specific immunity following stem cell transplant. The genetically modified T cells can re-expand in the host. In instances of GVHD, activation of iC9 with rimiducid leads to rapid killing of alloreactive T cells and resolution of GVHD.

The genetic modification will typically be performed at a specialist site, remote from the donor patient. Thus the cells need to be transported to the donor, which typically involves cryopreservation. The next example considers parameters which can affect the recovery and viability of the T cells before, during and after cryopreservation.

### Example 2: Characterization of Cryopreservation Process Parameters

Cell concentration and DMSO concentration have been identified as potentially having a key impact on the thawing cell viability and recovery, so these parameters were investigated in a DoE study. A Block factor, based on the statistical theory of arranging experimental units that are similar to one another, was added later to reduce the variability of the study. Using this theory, regressions that are different between the blocks would indicate experimental variations. Besides the primary factors the model also examines interaction between the 3 primary factors (Alias Terms).

The cell concentration range for the cells in the culture was 1-3×10⁶ cells/mL, and the DMSO content in final formulation was 2 to 8% (v/v).

### Cell Production and Preparation for Cryopreservation

Leukapheresis obtained from healthy donors were used as a starting material for this process. The cells were manufactured as described in Example 1. On harvest (Day 9), cells were formulated at three different concentrations: 1.3×10⁶/mL, 2.6×10⁶/mL and 3.6×10⁶/mL and diluted with CryoStor™ cell cryopreservation media CS10 (CS10) so that the final DMSO concentrations were 2%, 5%, or 8% (v/v). Following dilution cells were cryo-preserved using a controlled rate freezing (CRF).

### Post-Thaw and Post-Culture Measurements

The cryopreserved cells in vials were thawed in batches using a DryBath. Post-thaw cells were diluted 10× in PrimeXV+10% FBS complete medium. 300 μl aliquot was removed for cell count and viability measurements using NC-3000 Nucleocounter. The samples post-thaw were held for 60 min at 37° C. before centrifuging at 350×g for 10 min with deceleration set at 2. After centrifugation, the supernatant was removed by decantation and cells were resuspended in 5 mL of PrimeXV+10% FBS complete medium. Post-spin cell count and viability measurements were performed using NC-3000. The cells were placed in culture in an incubator at 37° C./5% CO₂. After 20±4 hours post-incubation, the cultures were supplemented with 200 IU/mL of IL-2 and cultures were continued for another 20±4 hours. After a total of 40±8 hours incubation, the cultures were harvested, and cell count and viability measurements were performed using NC-3000.

### Results

Cell viability ranged from 75.2-85.1%. The cell recovery was at least 97%.

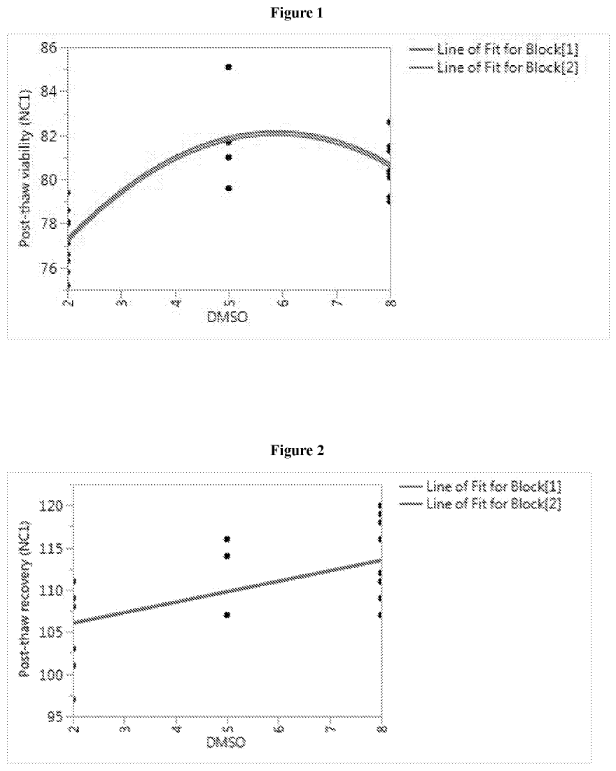

A first prediction profiler (FIG. 1), used to demonstrate multivariable effect, showed the DMSO concentrations impacted viability in a quadratic way with a desirable range between 5% and 7%, and the maximized viability at 5.88%. A second prediction profiler (FIG. 2) indicated that the recovery was in linear relationship with the DMSO in the tested concentrations indicating higher DMSO concentrations give better recovery.

This information about the effect of DMSO on viability and recovery can be combined with knowledge about its safety profile, and points to the optimum DMSO concentration being between 5-7% for cryopreservation. A concentration of about 5% provides a favourable balance of all three parameters, particularly in relation to safety for pediatric patients. A concentration below 5% is not favoured for cells which will be subjected to a cryopreservation procedure.

### Example 3: Cell Infusion

### Preparing Cells

Cells prepared according to Example 1 are washed in Plasma-Lyte A™. The supernatant is removed and the final volume is brought up to 7.5 mL using Plasma-Lyte A™ supplemented with HSA (Nova Biologics) to give a HSA concentration of 1% (v/v). 7.5 mL of CryoStor CS10™ (containing 10% v/v DMSO) is then added, to give a final volume of 15 mL. The final composition thus contains 0.5% HSA (v/v) and 5% DMSO (v/v). The cells are then cryopreserved for shipment.

### Thawing the Cells

The cryopreserved cells were prepared for infusion using a sterile water bath heated to 37°±1° C. To thaw the product bag, the ports of the final product bag are submerged into the 37°±1° C. water bath while visually inspecting the bag for potential leaks. If no leak on ports are observed or detected, the final product bag is submerged entirely in the 37°±1° C. water bath ensuring that water does not enter the zip-seal bag. The final product bag is then gently massaged during the thaw so that the thawed cell suspension circulates through the bag creating a more even temperature distribution of the thawed liquid in the bag. Just before the last piece of ice has thawed, the final product bag is removed from the water bath, removed from the zip-seal container and placed on a sterile drape.

### Infusion Preparation

A bag spike with needle free valve (or other sampling device) is inserted aseptically into the thawed final product bag and a Plasma-Lyte A™ bag. As an alternative, 0.9% NaCl solution can be used instead of Plasma-Lyte A™. 35 mL of Plasma-Lyte A™ (or 0.9% NaCl) is aseptically aspirated into a syringe. While gently mixing the product bag, the syringe's contents are slowly transferred into the product bag thus diluting the cells in the bag (from 15 mL to 50 mL). The empty syringe is then detached from the final product bag and the diluted final product is transported to the infusion location. The final product bag is then connected to a patient infusion line and infused over a period of 20-30 minutes.

To ensure that all cells in the bag are used, the final product bag is rinsed with an additional minimal volume of >15 mL of Plasma-Lyte A™ (or 0.9% NaCl) before the final product volume is drained from the bag. This rinse is performed by connecting a Plasma-Lyte A (or 0.9% NaCl) bag to the infusion set and draining the diluent into the final product bag by gravitational flow. As an alternative, a Plasma-Lyte A™ (or 0.9% NaCl) bolus can be connected to the needle free valve (or other sampling device) on the bag spike and injected into the bag.

It will be understood that the inventors' work has been described above by way of example only and modifications may be made while remaining within the scope and spirit of the invention.

1. A composition comprising T cells and serum albumin and dimethyl sulfoxide (DMSO), wherein the composition comprises <2.5% (v/v) serum albumin, and wherein DMSO is present at a ≥5-fold excess (v/v) compared to the serum albumin.

2. The composition of claim 1, comprising <2.2% (v/v), <2% (v/v), <1% (v/v), or about 0.5% (v/v) serum albumin.

3. The composition of claim 1 or claim 2, wherein DMSO is present at a 5-fold to 30-fold excess (v/v) compared to the serum albumin.

4. The composition of claim 3, wherein the excess is from 5-fold to 15-fold, from 6-fold to 12-fold, from 8-fold to 11-fold, or about 10-fold.

5. A composition comprising T cells, serum albumin, and DMSO, wherein the concentration of serum albumin (v/v) is from 0.1-1% and the concentration of DMSO (v/v) is 3-fold to 30-fold higher than the concentration of serum albumin.

6. The composition of claim 5, wherein the serum albumin concentration is <1% (v/v), and the DMSO concentration is from 8-fold to 11-fold higher than the serum albumin concentration.

7. The composition of claim 5 or claim 6, wherein concentration of DMSO is <10% (v/v).

8. The composition of claim 7, wherein the concentration of DMSO is from 2-8% (v/v), from 4-7% (v/v), from 5-6% (v/v), or about 5% (v/v).

9. A composition comprising T cells, serum albumin, and DMSO, wherein the concentration of serum albumin (v/v) is 0.1-1% and the concentration of DMSO is 5-7% (v/v).

10. The composition of claim 9, wherein the concentration of serum albumin (v/v) is between 0.2-0.8%, between 0.3-0.7%, between 0.4-0.6%, or about 0.5%.

11. The composition of any one of claims 1 to 10, having between 1×10⁶ and 40×10⁶ T cells/mL.

12. The composition of any one of claims 1 to 11, in cryopreserved form.

13. A composition comprising T cells and serum albumin, wherein the composition comprises about 0.15% (v/v) serum albumin.

14. The composition of claim 13, also including dimethyl sulfoxide (DMSO).

15. The composition of claim 14, wherein DMSO is present at a 3-fold to 30-fold excess (v/v) compared to the serum albumin.

16. The composition of claim 15, wherein the excess is from 3-fold to 15-fold, from 6-fold to 12-fold, from 8-fold to 11-fold, or about 10-fold.

17. A composition comprising T cells, serum albumin, and DMSO, wherein the concentration of serum albumin (v/v) is from 0.03-0.6% and the concentration of DMSO (v/v) is 3-fold to 30-fold higher than the concentration of serum albumin.

18. The composition of claim 17, wherein the serum albumin concentration is <0.3% (v/v), and the DMSO concentration is from 8-fold to 11-fold higher than the serum albumin concentration.

19. The composition of claim 17 or claim 18, wherein concentration of DMSO is <3% (v/v).

20. The composition of claim 7, wherein the concentration of DMSO is from 0.6-2.4% (v/v), from 0.8-2.2% (v/v), from 1.5-2% (v/v), or about 1.8% (v/v).

21. A composition comprising T cells and DMSO, wherein the composition comprises <0.15% (v/v) DMSO.

22. A composition comprising T cells, serum albumin, and DMSO, wherein the concentration of serum albumin is 0.03-0.3% (v/v) and the DMSO concentration is 0.15-0.20% (v/v).

23. The composition of any one of claims 13 to 22, having between 0.3×10⁶ and 12×10⁶ T cells/mL.

24. The composition of any preceding claim, wherein the T cells are genetically modified T cells, such as CAR-T cells.

25. The composition of any preceding claim, wherein the T cells are human T cells and the serum albumin is human serum albumin.

26. A process for preparing the composition of any one of claims 13 to 25, comprising the step of diluting a composition of any one of claims 1 to 12.

27. The process of claim 26, wherein dilution is performed with an electrolyte solution.

28. The process of claim 27, wherein the electrolyte solution is normal saline.

29. The process of claim 27, wherein the electrolyte solution is a sterile, nonpyrogenic, isotonic solution with a pH of 6.5-8.0 that has about 5.26 g/L NaCl, about 5.02 g/L sodium gluconate, about 3.68 g/L sodium acetate trihydrate, about 0.37 g/L KCl, and about 0.3 g/L MgCl₂.6H₂O.

30. A method of treating a subject, comprising a step of administering a composition of any one of claims 1 to 25 to a patient in need thereof.

31. A container comprising a suspension of T cells, serum albumin and dimethyl sulfoxide (DMSO), wherein the composition comprises <2.5% (v/v) serum albumin, and wherein DMSO is present at a ≥5-fold excess (v/v) compared to the serum albumin, and wherein the suspension is to be administered to a patient.

32. The container of claim 31, wherein the suspension comprises <2.2% (v/v), <2% (v/v), <1% (v/v), or about 0.5% (v/v) serum albumin.

33. The container of claim 31 or claim 32, wherein DMSO is present at a 5-fold to 30-fold excess (v/v) compared to the serum albumin.

34. The container of claim 33, wherein the excess is from 5-fold to 15-fold, from 6-fold to 12-fold, from 8-fold to 11-fold, or about 10-fold.

35. A container comprising a suspension of T cells, serum albumin, and DMSO, wherein the concentration of serum albumin (v/v) is from 0.1-1% and the concentration of DMSO (v/v) is 3-fold to 30-fold higher than the concentration of serum albumin, and wherein the suspension is to be administered to a patient.

36. The container of claim 35, wherein the serum albumin concentration is <1% (v/v), and the DMSO concentration is from 8-fold to 11-fold higher than the serum albumin concentration.

37. The container of claim 35 or claim 36, wherein concentration of DMSO is <10% (v/v).

38. The container of claim 37, wherein the concentration of DMSO is from 2-8% (v/v), from 4-7% (v/v), from 5-6% (v/v), or about 5% (v/v).

39. A container comprising a suspension of T cells, serum albumin, and DMSO, wherein the concentration of serum albumin (v/v) is 0.1-1% and the concentration of DMSO is 5-7% (v/v), and wherein the suspension is to be administered to a patient.

40. The container of claim 39, wherein the concentration of serum albumin (v/v) is between 0.2-0.8%, between 0.3-0.7%, between 0.4-0.6%, or about 0.5%.

41. The container of any one of claims 31 to 40, having between 1×10⁶ and 40×10⁶ T cells/mL.

42. The composition of any one of claims 31 to 41, in cryopreserved form.

43. A method of treating a subject, comprising a step of administering to a patient in need thereof the composition of any one of claims 13 to 25 by intravenous infusion.

44. The method of claim 43, wherein the intravenous infusion time is between 15 and 120 minutes.

45. The method of claim 43, wherein the intravenous infusion time is up to 30 minutes.

46. The method of any one of claims 41-43, wherein the infusion volume is between 50 and 100 mL.

47. The method of any one of claims 1-4, wherein the infusion volume is about 50 mL.

48. The composition, method, process, or container of any preceding claim, wherein the T cells comprise a polynucleotide that encodes a chimeric signaling polypeptide, wherein the chimeric signaling polypeptide comprises:
(i) a costimulatory polypeptide cytoplasmic signaling region;
(ii) a truncated MyD88 polypeptide region lacking the TIR domain;
(iii) a truncated MyD88 polypeptide region lacking the TIR domain and a costimulatory polypeptide cytoplasmic signaling region; or
(iv) a truncated MyD88 polypeptide region lacking the TIR domain and a CD40 cytoplasmic polypeptide region lacking the CD40 extracellular domain.

49. The composition, method, process, or container of claim 48, wherein costimulatory polypeptide cytoplasmic signaling region is a signaling region that activates the signaling pathways activated by MyD88, CD40 and/or MyD88-CD40 fusion chimeric polypeptide.

50. The composition, method, process, or container of any of the preceding claim, wherein the T cells comprises a polynucleotide encoding an inducible chimeric pro-apoptotic polypeptide.

51. A kit comprising:
(a) a container comprising an interior space; and
(b) a composition contained within the interior space, wherein the composition comprises:
(i) T cells in an amount between about 1×10⁶ and 40×10⁶ (e.g., about 5×10⁶) cells/ml;
(ii) serum albumin in a concentration of about 0.1%-0.6% v:v;
(iii) DMSO in a concentration of about 5%-7% v:v; and
(iv) a pharmaceutically acceptable carrier for human injection.

52. The kit of claim 51, wherein the container comprises a bag, e.g., a cryopreservation bag.

53. The kit of claim 52, wherein the container further comprises a bag spike in fluidic communication with the interior space.

54. The kit of claim 51, wherein the T cells are genetically engineered.

55. The kit of claim 51, wherein the serum albumin is human serum albumin.

56. The kit of claim 51, wherein the serum albumin is present in concentration of about 0.5% v:v.

57. The kit of claim 51, wherein DMSO is present in a concentration of about 5% v:v.

58. The kit of claim 51, wherein the pharmaceutically acceptable carrier comprises sodium chloride, sodium gluconate sodium, acetate trihydrate, potassium chloride, and magnesium chloride

59. The kit of claim 51, wherein the pharmaceutically acceptable carrier comprises Pharma Lyte or Plasma Lyte.

60. The kit of claim 51, wherein the composition has a volume of about 15 ml.

61. A kit comprising:
(a) a container comprising an interior space; and
(b) a composition contained within the interior space, wherein the composition comprises:
(i) T cells in an amount between about 15×10⁶ cells and about 600×10⁶ (e.g., about 75×10⁶) cells;
(ii) serum albumin in an amount between about 0.03% and about 1.8% serum albumin, e.g., about 1.5% v:v;
(iii) DMSO in a concentration of about 1.5%-2.1 v:v (e.g., about 1.5% v:v); and
(iv) a pharmaceutically acceptable carrier for human injection.

62. The kit of claim 61, wherein the container comprises a bag, optionally including a tube comprising a bag spike, wherein the tube is in fluidic communication with the interior space.

63. The kit of claim 61, wherein the composition has a volume of about 50 ml.

64. A composition as described herein for use in treating a disease, e.g., cancer.

65. A use of a composition as described herein for treating a disease, e.g., cancer.

66. A use of a composition as described herein in the preparation of a medicament for treating a disease, e.g., cancer.
